(19) United States
(12) Patent Application Publication
(10) Pub. No.: US 2017/0189312 A1
(43) Pub. Date: Jul. 6, 2017

(54) COMPOSITIONS CONTAINING POLYCARBODIIMIDES AND AMINO COMPOUNDS FOR ENHANCED COLOR RETENTION AND DURABILITY

(71) Applicant: L'OREAL, Paris (FR)

(72) Inventors: Nghi VAN NGUYEN, Clark, NJ (US); Charles Michael Sanford SHAW, Clark, NJ (US); Jim Mitchell SINGER, Clark, NJ (US); Daniella GONZALEZ-TORO, Clark, NJ (US); Andrea Michelle ELSEN-WAHRER, Clark, NJ (US)

(21) Appl. No.: 15/395,484

(22) Filed: Dec. 30, 2016

Related U.S. Application Data
(63) Continuation of application No. 14/986,114, filed on Dec. 31, 2015.
Application No. 14/986,114 is a continuation of application No. 14/986,283, filed on Dec. 31, 2015.
Application No. 14/986,283 is a continuation of application No. PCT/US2016/069472, filed on Dec. 30, 2016.

(51) Int. Cl. A61K 8/88; A61Q 5/00; A61Q 5/10; A61K 8/81; A61K 8/58

(52) U.S. Cl. CPC A61K 8/88; A61K 8/817; A61K 8/585; A61Q 5/10; A61Q 5/004; A61K 2800/884; A61K 2800/882; A61K 2800/43

(57) ABSTRACT

Compositions including polycarbodiimide, and derivatives thereof, together with optional colorants, and amino compounds and derivatives thereof to enhance the quality of keratinous substrates are disclosed. The present invention relates to a cosmetic treatment and process for treating the keratinous substrates, in particular for hair coloration and treatment of artificially colored hair, wherein the composition can include the polycarbodiimide and the amino compound in amounts sufficient to impart one or more of improved color deposit, improved color retention and extended color durability.

### CROSS-REFERENCE TO RELATED APPLICATION

This patent application claims the benefit of U.S. Non-Provisional patent application Ser. No. 14/986,283 filed on Dec. 31, 2015, and entitled “COMPOSITIONS CONTAINING POLYCARBODIIMIDES AND AMINO COMPOUNDS FOR TREATING KERATINOUS SUBSTRATES,” and U.S. Non-Provisional patent application Ser. No. 14/986,114 filed on Dec. 31, 2015, and entitled “COMPOSITIONS CONTAINING POLYCARBODIIMIDES AND AMINO SILICONE COMPOUNDS FOR TREATING KERATINOUS SUBSTRATES,” the disclosures of which are incorporated by reference as if fully rewritten herein.

### FIELD OF THE INVENTION

The present invention generally relates to compositions and methods for treating keratinous substrates. More particularly, the present invention relates to keratinous treatment compositions comprising at least one polycarbodiimide compound, optionally, a colorant or dye, and an amino compound chosen from polyamines and silicone amines, for one or more of enhanced color deposit, color retention, and color fade resistance to shampoo, washing, and to other hair treatments.

### BACKGROUND OF THE INVENTION

There are many products available for changing the natural color of hair. The process of changing the color of hair can involve either depositing an artificial color onto the hair, which provides a different shade or color to the hair, or lifting the color of the hair, such as for example, from a dark brown shade to a medium brown or a light brown shade. Hair color can be changed using permanent, semi-permanent, or temporary hair coloring products.

Many consumers desire a permanent color change or long lasting color and therefore use products containing permanent dyes. Conventional permanent hair coloring products are dye compositions comprising oxidation dye precursors, which are also known as primary intermediates or couplers. These oxidation dye precursors are colorless or weakly colored compounds which, when combined with oxidizing products, give rise to colored complexes by a process of oxidative condensation. The oxidizing products conventionally use peroxides such as hydrogen peroxide as oxidizing agents. Such permanent hair color products also contain ammonia or other alkalizing agents such as monoethanolamine (MEA) which causes the hair shaft to swell, thus allowing the small oxidative dye molecules to penetrate the cuticle and cortex before the oxidation condensation process is completed. The resulting larger-sized colored complexes from the oxidative reaction are then trapped inside the hair fiber, thereby permanently altering the color of the hair.

Newly, permanently colored hair usually has a vibrant, shiny, and rich appearance. Unfortunately, however, in just a few short weeks, or in some cases even less time, the color begins to fade due to washing or exposure to environmental conditions. For instance, gorgeous rich brown colors become muddy and dull, beautiful shades of blonde turn brassy, and vibrant reds do not look so vibrant anymore acquiring golden, orange or brownish tonalities not desirable to the consumer. As described herein, the inventors of the instant disclosure have developed compositions and methods that confer one or more benefits including improved color deposit, improved color retention and extended color durability.

### BRIEF SUMMARY OF THE INVENTION

In an exemplary embodiment, a keratinous treatment composition including a polycarbodiimide compound, optionally a colorant or dye, and an amino compound chosen from polyamines and silicone amines is disclosed. The composition includes about 0.1 to about 40.0%, by weight, based on the total weight of the composition, of a combined amount of the polycarbodiimide compound and the amino compound. The composition includes amounts of each of the polycarbodiimide compound and the amino compound chosen from polyamines and silicone amines sufficient to impart one or more of improved color deposit, improved color retention and extended color durability.

In some embodiments, the composition comprises a combined amount of the polycarbodiimide compound and the amino compound chosen from polyamines and silicone amines from about 0.25 to about 20%, by weight, based on the total weight of the composition, and in some embodiments, from about 1% to about 10%, and in some embodiments, from about 1% to about 5%, by weight, based on the total weight of the composition.

In another exemplary embodiment, a method of treating a keratinous substrate chosen from hair, eyelashes and eyebrows includes applying to the keratinous substrate a composition including the polycarbodiimide and the amino compound chosen from polyamines and silicone amines in an amount effective to impart one more of improved color deposit, improved color retention and extended color durability; wherein the polycarbodiimide compound is present at a concentration of from about 0.01 to about 20% by weight, based on the total weight of the composition.

In some particular embodiments, the composition comprises a dye or colorant together with a combined amount of the polycarbodiimide compound and the amino compound chosen from polyamines from about 0.1% to about 40%, by weight, based on the total weight of the composition, and in some embodiments, from about 0.25 to about 20%, and in some embodiments from about 1% to about 10%, and in some embodiments, from about 1% to about 5%, by weight, based on the total weight of the composition. According to such embodiments, the polyamine may be chosen from polyvinylamines.

In some particular embodiments, the composition comprises a combined amount of the polycarbodiimide compound and the amino compound chosen from silicone amines from about 0.1% to about 40%, by weight, based on the total weight of the composition, and in some embodiments, from about 0.25 to about 20%, and in some embodiments from about 1% to about 10%, and in some embodiments, from about 1% to about 5%, by weight, based on the total weight of the composition. According to such embodiments, the silicone amine may be chosen from side chain silicones. In a particular embodiment, the silicone amine may be amodimethicone.

The present invention is also directed to a method for cosmetic treatment of keratinous tissues, such as keratinous fibers, by applying to a surface of the keratinous tissue, such as the cuticle of hair fibers a composition comprising polycarbodiimide compound, optionally a colorant or dye, and an amino compound chosen from polyamines and silicone amines.

In some embodiments, the composition is applied together with color treatment. In some such embodiments, the composition comprises a colorant or dye, and an amino compound chosen from polyamines.

In some embodiments, the composition is applied at a time following color treatment. In some such embodiments, the composition comprises an amino compound chosen from silicone amines.

In some embodiments, the composition is applied at a time preceding color treatment.

In some embodiments, the keratinous substrate in the above-described composition is heated and the composition is applied to the substrate before heating or during heating or after heating the substrate.

The present invention is also directed to methods and kits for cosmetic treatment of keratinous tissues, such as keratinous fibers, by applying the above-disclosed composition onto a surface of the keratinous tissue, such as the cuticle of hair fibers in a stepwise fashion. According to some such embodiments, the kit includes separate packaging of one or more of the actives of the inventive composition provided in one or more of thickened or un-thickened aqueous and non-aqueous phases, and packaging of any of the foregoing with one or more of processing agents selected from a coloring agent, a pigmenting agent, a permanent process agent, a relaxing process agent, a straightening process agent, and a highlighting process agent.

Other features and advantages of the present invention will be apparent from the following more detailed description of some embodiment which illustrates, by way of example, the principles of the invention.

### DETAILED DESCRIPTION OF THE INVENTION

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“Active material” or “weight” as used herein with respect to the percent amount of an ingredient or raw material, refers to 100% activity of the ingredient or raw material. All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

As used herein, the terms “applying a composition onto keratin fibers” and “applying a composition onto hair” and variations of these phrases are intended to mean contacting the fibers or hair, with at least one of the compositions of the invention, in any manner.

“At least one,” as used herein, means one or more and thus includes individual components as well as mixtures/combinations.

“Color deposit” refers to refers to a visually distinguishable color effect imparted to a keratinous substrate by the application of a composition including a colorant or dye to the substrate, resulting in permanent, ie, not “wash out” or “try on” color application or composition.

“Color retention” and “extended color durability” refer to the maintenance of visibly distinguishable color effect imparted to a keratinous substrate from an application of a composition including a colorant or dye to the substrate, and in particular, increased maintenance of the color effect using compositions according to the disclosure as comparted with comparative compositions that lack the inventive combinations of polycarbodiimide and one or more of amino compounds selected from polyamines and silicone amines.

The term “comprising” (and its grammatical variations) as used herein is used in the inclusive sense of “having” or “including” and not in the exclusive sense of “consisting only of.”

“Heating” refers to the use of elevated temperature (i.e., above room temperature such as at or above 40° C.). In one embodiment, the heating in the inventive method may be provided by directly contacting the at least one keratinous fiber with a heat source, e.g., by heat styling of the at least one keratinous fiber. Non-limiting examples of heat styling by direct contact with the at least one keratinous fiber include flat ironing and curling methods using elevated temperatures (such as, for example, setting hair in curlers and heating, and curling with a curling iron and/or hot rollers). In another embodiment, the heating in the inventive method may be provided by heating the at least one keratinous fiber with a heat source which may not directly contact the at least one keratinous fiber. Non-limiting examples of heat sources which may not directly contact the at least one keratinous fiber include blow dryers, hood dryers, heating caps and steamers.

“A heat-activated” composition, as used herein, refers to a composition which, for example, shapes the at least one keratinous fiber better than the same composition which is not heated during or after application of the composition.

Another example includes a composition that retains a shape of at least one keratinous fiber better than the same composition that is not heated during or after application.

“High humidity,” as defined herein, refers to atmospheric humidity above 40%.

The term “wash cycle” as used herein, refers to a step or process of washing a keratinous substrate and may include treating the substrate with a surfactant-based product (e.g., shampoo or conditioner or body wash) then washing or rinsing the substrate with water. The term “wash cycle” may also include washing or rinsing the substrate with water.

“Homogeneous” means having the visual appearance of being substantially uniform throughout, i.e., visually appears as a single-phase emulsion and/or dispersion.

“Keratinous substrate,” as used herein, includes, but is not limited to, skin, hair, and nails. “Keratinous substrate” as used herein also includes “keratinous tissue” or “keratinous fibers,” which as defined herein, may be human keratinous fibers, and may be chosen from, for example, hair, such as hair on the human head, or hair comprising of eyelashes or hair on the body.

The term “treat” (and its grammatical variations) as used herein refers to the application of the compositions of the present invention onto keratinous substrates such as keratinous fibers or hair or skin.

The term “altering the color” and variations thereof as used herein may refer to dyeing or coloring hair or depositing color onto the hair.

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

It has been surprisingly and unexpectedly discovered by the inventors that compositions comprising a combination at least one polycarbodiimide compound and at least one amino compound chosen from polyamines and silicone amines, particularly compositions wherein the amino compound is selected from silicone amines, when applied in combination with a colorant or dye compound to keratinous substrates such as hair, with and without heat, deposit color more effectively as compared to traditional or commercial hair dyes using similar or less amounts of dye compounds. It has also been surprisingly and unexpectedly discovered by the inventors that compositions as described, particularly compositions wherein the amino compound is selected from polyamines, when applied prior to application of color, provide for lasting retention of color in treated hair, even after repeated washings and other post color treatments. Thus, the compositions and methods of the present invention can provide for enhanced color visibility and coverage and color retention and durability.

Without being bound to any one theory, the inventors of the present disclosure believe that the polycarbodiimide and amino compounds in the keratinous treatment compositions of the invention react to each other and to the keratin substrate when such compositions are applied onto keratinous substrates such as hair or skin to form a crosslinked network of polymers. It is further believed that the crosslinked compositions provide a protective barrier useful in cosmetic applications, in particular but not limited to hair cosmetics, such that the hydrophobicity of the keratinous substrates is improved or restored resulting in the observed performance as reported herein, and one or more of significantly improved color deposit, improved color retention and extended color durability.

The compositions, according to the invention, are compositions including at least one or more of each of polycarbodiimide and amino compounds, and optionally one or more dyes or colorants. The composition may include other suitable ingredients for hair treatment or hair repair. For example, known solvents and/or additives may be utilized in addition to the polycarbodiimide compound and amino compound chosen from polyamines and silicone amines to provide additional benefits to the composition.

Compositions Comprising One or More of Each of Polycarbodiimide and Amino Compounds Selected from Polyamines and Silicone Amines

In accordance with the various embodiments, the combination of polycarbodiimide compound and the amino compound chosen from polyamines and silicone amines is present in the compositions according to the disclosure in amounts range from about 0.1% to about 40%, and in some embodiments from about 0.25% to about 20%, and in some further embodiments from about 1% to about 10%. In some representative embodiments, the combination of polycarbodiimide compound and the amino compound chosen from polyamines and silicone amines is present from about 0.5% to about 10%, and from about 0.5% to about 5%. In yet other embodiments, the combination of polycarbodiimide compound and the amino compound chosen from polyamines and silicone amines is present from at least 0.5%. And in yet other embodiments, the combination of polycarbodiimide compound and the amino compound chosen from polyamines and silicone amines is present from at least 0.5% to up to about 40%. In some specific embodiments, the combination of polycarbodiimide compound and the amino compound chosen from polyamines and silicone amines is present at about 5%. In some other specific embodiments, the combination of polycarbodiimide compound and the amino compound chosen from polyamines and silicone amines is present at about 2.5%. In some other specific embodiments, the combination of polycarbodiimide compound and the amino compound chosen from polyamines and silicone amines is present at about 0.75%.

Thus, the combination of polycarbodiimide compound and the amino compound chosen from polyamines and silicone amines may be present from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 to about 40 percent by weight, including increments and ranges therein and there between.

The range of the weight ratio of the polycarbodiimide to the amino compound chosen from polyamines and silicone amines in the composition over which their association provides hydrophobicity to keratinous substrates such as hair, is from about 10:1 to about 1:10, including all ranges and subranges therebetween.

In various embodiments, the combination of the polycarbodiimide compound and the amino compound chosen from polyamines and silicone amines is present in the compositions in the foregoing amounts, wherein the ratio of the polycarbodiimide compound to the amino compound chosen from polyamines and silicone amines ranges from about 10:1 to about 1:10. In some exemplary embodiments, the ratio of the polycarbodiimide compound to the amino compound chosen from polyamines and silicone amines is from about 2:1 to 1:2. In some exemplary embodiments, the ratio of the polycarbodiimide compound to the amino compound chosen from polyamines and silicone amines is from about 1:1. In some other exemplary embodiments, the ratio of the polycarbodiimide compound to the amino compound chosen from polyamines and silicone amines is from about 1:2.

In some embodiments, compositions according to the disclosure comprise, in addition to the polycarbodiimide and amino compounds selected from polyamines and silicone amines, additional optional compounds as more fully described herein. In some particular embodiments, the compositions comprise at least one or more additional compounds selected from polymeric (for example, cellulosic) based thickeners, amphoteric surfactants, nonionic fixing polymers, cationic surfactants, polyols, and propellants.

Polycarbodiimide Compounds

A carbodiimide group is a linear triatomic moiety generally depicted by Formula (I):

*—(N═C═N)—* (I)

At least one of the nitrogens is linked to or incorporated into a backbone or other bridging group to result in a molecule having at least two carbodiimide groups.

Polycarbodiimides

In one embodiment, the polycarbodiimides comprising of at least two carbodiimide units, as described above, can be represented by Formula (II):

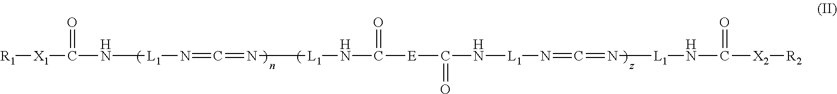

wherein X₁ and X₂ each independently represent O, S or NH. R₁ and R₂ are selected from a hydrocarbon group containing one or more catenary or non-catenary hetero-atoms, such as nitrogen, sulfur and oxygen, and linear or branched and cyclic or acyclic groups which can be ionic or non-ionic segments, or a partially or fully fluorinated hydrocarbon group that may contain one or more catenary or non-catenary hetero-atoms; n and z are, each independently, an integer of 0 to 20; L₁ (Linker of carbodiimide groups) is selected from a C₁ to C₁₈ divalent aliphatic hydrocarbon group, a C₃ to C₁₃ divalent alicyclic hydrocarbon group, a C₆ to C₁₄ divalent aromatic hydrocarbon group, and a C₃ to C₁₂ divalent heterocyclic group; wherein a plurality of L₁s may be identical to or different from one another, and wherein in another embodiment, L₁ of formula (II) is selected from a C₁ to C₁₈ divalent aliphatic hydrocarbon group, a C₃ to C₁₃ divalent alicyclic hydrocarbon group, a C₆ to C₁₄ divalent aromatic hydrocarbon group that is not chosen from m-tetramethylxylylene, and a C₃ to C₁₂ divalent heterocyclic group; wherein a plurality of L₁s may be identical to or different from one another;

wherein E is a radical selected from the following formulas:

O—R₃—O;S—R₄—S; and

R₅—N—R₄—N—R₅;

wherein R₃ and R₄ are each independently hydrocarbon radicals that may contain halogen atoms or one or more catenary (i.e.; in chain, bonded only to carbon) or non-catenary hetero atoms, including an aromatic, cycloaliphatic, aryl and alkyl radical (linear or branched) and R₅ is hydrogen, or a hydrocarbon radical which can contain halogen atoms or one or more catenary (i.e.; in chain, bonded only to carbon) or non-catenary hetero atoms.

Examples of R₁ and R₂ can be methyl glycolate, methyl lactate, polypropylene glycol, polyethylene glycol monomethyl ether, dialkylamino alcohol.

Examples of L₁ can be the diradical of tolylene, hexamethylene, hydrogenated xylylene, xylylene, 2,2,4-trimethylhexamethylene, 1,12-dodecane, norbornane, 2,4-bis-(8-octyl)-1, 3-dioctylcyclobutane, 4,4′-dicyclohexylmethane, tetramethylxylylene, isophorone, 1,5-naphthylene, 4, 4′ diphenylmethane, 4, 4′ diphenyldimethylmethane, phenylene.

In accordance with the various embodiments, the polycarbodiimide compound is present in the compositions according to the disclosure in amounts range from about 0.01% to about 20%, and in some embodiments from about 0.1% to about 15%, and in some further embodiments from about 0.1% to about 10%. In some representative embodiments, the polycarbodiimide compound is present from about 0.1% to about 5%, and from about 0.2% to about 3%. In yet other embodiments, the polycarbodiimide compound is present from at least 0.2%. And in yet other embodiments, the polycarbodiimide compound is present from at least 0.2% to up to about 40%. In some specific embodiments, the polycarbodiimide compound is present at about 1.5%. In some specific embodiments, the polycarbodiimide compound is present at about 0.75%. In some other specific embodiments, the polycarbodiimide compound is present at about 0.25%.

Thus, the polycarbodiimide compound may be present from about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, to about 20 percent by weight, including increments and ranges therein and there between.

Polycarbodiimides may include polymers with a plurality of carbodiimide groups appended to the polymer backbone. For example, U.S. Pat. No. 5,352,400 (the disclosure of which is incorporated by reference herein for all purposes as if fully set forth) discloses polymers and co-polymers derived from alpha-methylstyryl-isocyanates. Such a polymer is illustrated in Formula (III).

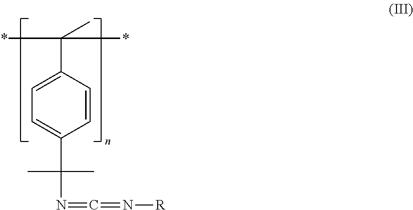

wherein R is an alkyl, cycloalkyl or aryl group (in some particular embodiments having from 1 to 24 carbon atoms).

In another embodiment, polycarbodiimides, according to the present disclosure, include polycarbodiimides having branched structures, like that shown in Formula (IV), and as described in Chapter 8 of Technology for Waterborne Coatings, E. J. Glass Ed., ACS Symposium 663, 1997; The Application of Carbodiimide Chemistry to Coating, by J. W. Taylor and D. R. Bassett (the disclosure of which is incorporated by reference herein for all purposes as if fully set forth).

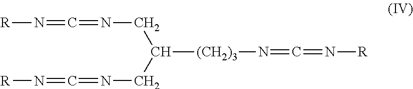

wherein R is an alkyl, cycloalkyl or aryl group (in some particular embodiments having from 1 to 24 carbon atoms).

In one embodiment, the compositions of the present disclosure do not employ a polycarbodiimide having a linker L₁ chosen from m-tetramethylxylylene.

Suitable polycarbodiimide compounds include, but are not limited to; those commercially sold by the suppliers Nisshinbo, Picassian, and 3M. Particularly suitable polycarbodiimide compounds include, but are not limited to, those known by the name under the CARBODILITE series, V-02, V02-L2, SV-02, E-02, V-10, SW-12G, E-03A, commercially sold by Nisshinbo.

In some embodiments, the polycarbodiimide of the present disclosure is selected from compounds of formula (II) wherein L₁ (Linker of carbodiimide groups) represents a C₁ to C₁₈ divalent aliphatic hydrocarbon group, a C₃ to C₁₃ divalent alicyclic hydrocarbon group, a C₃ to C₁₂ divalent heterocyclic group, or a C₆ to C₁₄ divalent aromatic hydrocarbon group; wherein a plurality of L₁s may be identical to or different from one another.

In other embodiments, the polycarbodiimide of the present disclosure is selected from compounds of formula (II) wherein L₁ is not chosen from m-tetramethylxylylene.

In certain embodiments, a composition of the present disclosure is devoid of a polycarbodiimide that has a linker L₁ chosen from m-tetramethylxylylene.

Amino Compounds Comprising Polyamines and Silicone Amines

The composition according to the present disclosure comprises one or more amino compounds selected from polyamines and silicone amines (also referred to as amino silicones). The term “amino compound” is intended to mean any compound comprising at least one primary, secondary or tertiary amine or a quaternary ammonium group.

In certain embodiments, the amino compounds of the present disclosure do not contain silicon atoms or silicone moieties. In other embodiments, the amino compounds of the present disclosure contain silicon atoms or silicone moieties.

Alkoxylated Polyamines

The alkoxylated polyamines of the present disclosure are chosen from amino compounds having at least two amino groups and at least one degree of alkoxylation. The alkoxylation is provided by an alkylene oxide group which is in some embodiments chosen from ethylene oxide and propylene oxide.

Non-limiting some examples of suitable alkoxylated polyamines include compounds corresponding to formula (IB):

NH₂R(R′CHCH₂O)ₓ(R′CHCH₂O)_{y}(R′CHCH₂I)_{z}RNH₂ (IB)

wherein R represents a —CH2-, —CH₂CH₂—, —CHCH₃— or —C(CH₃)₂— group, or a hydrocarbon radical containing at least 3 carbon atoms that is linear or branched, acyclic or cyclic, saturated or unsaturated, aliphatic or aromatic, substituted or unsubstituted;

x, y, and z independently of one another, represent numbers of from 0 to about 100;

R′ represents hydrogen, or an alkyl group, in some embodiments a methyl group; and

the sum of x+y+z is at least 1.

In formula (IB), R is in some embodiments a linear or branched, acyclic alkyl or alkenyl group or an alkyl phenyl group; x, y, and z independently of one another, in some embodiments represent numbers ranging from 2 to 100.

Examples of the alkoxylated polyamines for use in the present disclosure which correspond to formula (IB) include, for example, tetradecyloxypropyl-1,3-diaminopropane; a C12-14 alkyl oxypropyl-1,3-diaminopropane; a C12-15 alkyloxypropyl amine and other similar materials that are commercially available from Tomah under the tradename of TOMAH DA-17.

Other examples of alkoxylated polyamines of Formula (IB) are diamine compounds belonging to the JEFFAMINE series such as the JEFFAMINE D and JEFFAMINE ED series available from Huntsman Corporation, Salt Lake City, Utah. Examples of these Jeffamine compounds are JEFFAMINE D230, JEFFAMINE D400, JEFFAMINE D2000, JEFFAMINE D4000, JEFFAMINE HK-511, JEFFAMINE ED600, JEFFAMINE ED900, and JEFFAMINE ED2003. JEFFAMINE D series compounds are amine terminated PPGs (polypropylene glycols) and JEFFAMINE ED series compounds are polyether diamine based with a predominantly PEG (polyethylene glycol) backbone.

Other non-limiting some examples of suitable alkoxylated polyamines in the diamine form include compounds corresponding to formula (IIB):

NH₂(CH₂)ₓOCH₂CH₂O(CH₂)ₓNH₂ (IIB)

wherein x is 2 or 3.

Examples of alkoxylated polyamines of Formula (IIB) are diamine compounds belonging to the JEFFAMINE series available from Huntsman Corporation, Salt Lake City, Utah, such as JEFFAMINE EDR148, and JEFFAMINE EDR176.

Additional non-limiting some examples of alkoxylated polyamines in the triamine form include compounds corresponding to formula (IIIB):

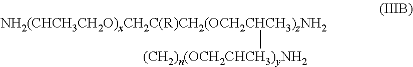

wherein R is hydrogen, —CH₂ or —C₂H₅,

n=0 or 1, and

x, y, and z independently of one another, represent numbers of from 0 to 100 and the sum of x+y+z is at least 1.

Examples of alkoxylated polyamines for use in the present disclosure which correspond to formula (IIIB) are triamine compounds belonging to the JEFFAMINE series such as the JEFFAMINE T series available from Huntsman Corporation, Salt Lake City, Utah. Examples of the JEFFAMINE T series compounds are JEFFAMINE T403, JEFFAMINE T3000, and JEFFAMINE T5000. JEFFAMINE T series compounds are triamines made by reacting PO with a triol initiator followed by aminating the terminal hydroxyl groups.

Another type of some alkoxylated polyamines include compounds of formulas (IVB) and (VB) hereunder:

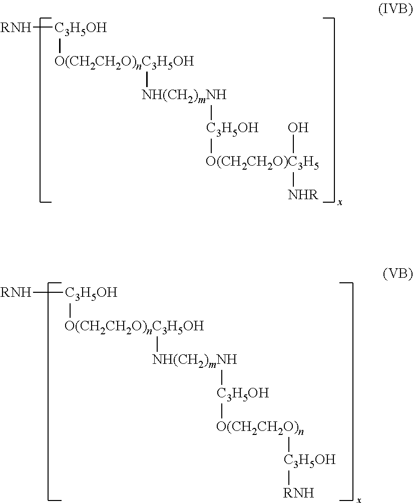

wherein

R in formula (IVB) represents the alkyl group derived from tallow and R in formula (VB) represents the alkyl group derived from coconut oil;

n in both formulas (IVB) and (VB) has a total value ranging from 10 to 20;

m in both formulas (IVB) and (VB) has a value ranging from 2 to 6; and

x in both formulas (IVB) and (VB) has a value ranging from 2 to 4.

One particular triamine alkoylated polyamine compound is JEFFAMINE T-500 polyetheramine of the formula:

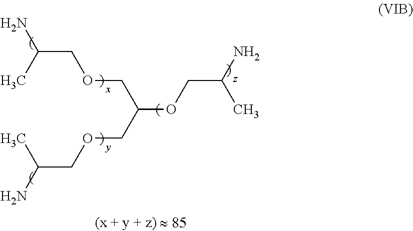 [SMILES: CC(N)COCC(COCC(C)N)OCC(C)N]

Other some types of alkoxylated polyamines include aminosilicones with at least one degree of alkoxylation.

Some examples of alkoxylated polyamines for use in the present disclosure include compounds of Formulas (IVB), (VB) and (VIB) above, such as PEG-15 Tallow Polyamine, PEG-15 Cocopolyamine, and JEFFAMINE T-500 polyetheramine, respectively.

Polyamines

The polyamines may in particular be aminated polysaccharides, amine substituted polyalkylene glycols, amine substituted polyacrylate crosspolymers, amine substituted polyacrylates, amine substituted polymethacrylates, proteins, protein derivatives, amine substituted polyesters, polyamino acids, polyalkylamines, diethylene triamine, triethylenetetramine, spermidine, spermine and mixtures thereof. The polyamines for use in the present disclosure can also be chosen from aminosilicones having at least two amino groups.

The amino compound of the present disclosure selected from polyamines may be, in particular having a weight-average molecular weight ranging from 500 to 1,000,000, in some embodiments ranging from 500 to 500,000, and preferentially ranging from 500 to 100,000. As amine-comprising polymer, use may be made of polyamines such as poly((C2-C5) alkyleneimines), and in particular polyethyleneimines and polypropyleneimines, especially poly(ethyleneimine)s; poly(allylamine); polyvinylamines and copolymers thereof, in particular with vinylamides; polyamino acids which have NH₂ groups; aminodextran; amino polyvinyl alcohol; acrylamidopropylamine-based copolymers; and chitosans.

The polyamines of the present disclosure may also be chosen from Vinylamine/Vinyl Alcohol Copolymer (INCI name).

Other some polyamines of the present disclosure include are amine substituted polyalkylene glycols such as PEG-15 cocopolyamine and PEG-15 Tallow Polyamine and amine substituted polyacrylate crosspolymer such as the product sold under the name CARBOPOL AQUA CC polymer by Lubrizol Advanced Materials, Inc.

The polyamine compound of the present disclosure may also be chosen from proteins and protein derivatives such as wheat protein, soy protein, oat protein, collagen, and keratin protein.

In an embodiment of the present disclosure, the polyamine compound is chosen from polyamino acid compounds comprising lysine, compounds comprising arginine, compounds comprising histidine, and compounds comprising hydroxylysine. Non limiting examples include chitosan and polyamino acids such as polyarginine, polyhistidine, polylysine, and mixtures thereof.

The polyamines of the present disclosure are also in some embodiments polyvinylamines which are generally sold under the trade name LUPAMINE or LUVIQUAT from BASF. In one some embodiment of the present disclosure, amino compound of the present disclosure is such as Vinylamine/vinylformamide copolymer (INCI name), sold as LUVIQUAT 9030 by BASF; alkoxylated polyamines which correspond to formula (IIIB) such as JEFFAMINE T403, JEFFAMINE T3000, and JEFFAMINE T5000 (in particular, corresponds to formula (VIB); alkoxylated monoamines which correspond to formula (IIIC) such as JEFFAMINE M-600; and alkyl amines selected from aliphatic amines and amidoamines; and mixtures, thereof.

In accordance with the various embodiments, the amino compound chosen from polyamines is present in the compositions according to the disclosure in amounts range from about 0.01% to about 40%, and in some embodiments from about 0.01% to about 20%, and in some further embodiments from about 0.1% to about 15%. In some representative embodiments, the amino compound chosen from polyamines is present from about 0.25% to about 10%, and from about 0.5% to about 5%. In yet other embodiments, the amino compound chosen from polyamines is present from at least 0.5%. And in yet other embodiments, the amino compound chosen from polyamines is present from at least 0.5% to up to about 40%. In some specific embodiments, the amino compound chosen from polyamines is present at about 1.5%. In some other specific embodiments, the amino compound chosen from polyamines is present at about 0.75%. In some other specific embodiments, the amino compound chosen from polyamines compound is present at about 0.25%.

Thus, the amino compound chosen from polyamines may be present from about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 to about 40 percent by weight, including increments and ranges therein and there between.

In accordance with some particular embodiments, the polyamines are selected from polyvinylamines.

Amino Silicone Compounds

In some embodiments, the composition according to the invention comprises one or more amino silicone compounds. The term “amino silicone compound” is intended to mean any silicone comprising at least one primary, secondary or tertiary amine or a quaternary ammonium group.

As amino silicone compounds that may be used in the scope of the invention, the following can be cited:

polysiloxanes corresponding to formula (A):

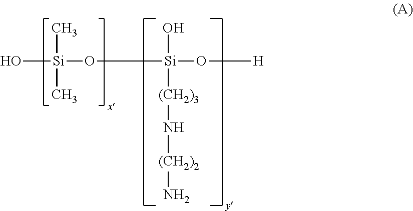

in which x′ and y′ are integers such that the weight-average molecular weight (Mw) is comprised between about 5000 and 500,000;

b) amino silicone compounds corresponding to formula (B):

R′ₐG3-a-Si(OSiG2)ₙ—(OSiGbR′2-b)ₘ-O-SiG3-a-R′ₐ (B)

in which:

G, which may be identical or different, designate a hydrogen atom, or a phenyl, OH or C1-C8 alkyl group, for example methyl, or C1-C8 alkoxy, for example methoxy,

a, which may be identical or different, denote the number 0 or an integer from 1 to 3, in particular 0;

b denotes 0 or 1, and in particular 1;

m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;

R′, which may be identical or different, denote a monovalent radical having formula —CqH2qL in which q is a number ranging from 2 to 8 and L is an optionally quaternized amino group chosen from the following groups:

—NR″-Q-N(R″)₂

—N(R″)₂

—N+(R″)₃A-

—N+H(R″)₂A-

—N+H₂(R″) A-

—N(R″)-Q-N+R″H₂A-

—NR″-Q-N+(R″)₂H A-

—NR″-Q-N+(R″)₃A-,

in which R″, which may be identical or different, denote hydrogen, phenyl, benzyl, or a saturated monovalent hydrocarbon-based radical, for example a C1-C20 alkyl radical; Q denotes a linear or branched CrH2r group, r being an integer ranging from 2 to 6, in some embodiments from 2 to 4; and A- represents a cosmetically acceptable ion, in particular a halide such as fluoride, chloride, bromide or iodide.

A group of amino silicone compounds corresponding to this definition (B) is represented by the silicones called “trimethylsilylamodimethicone” having formula (C):

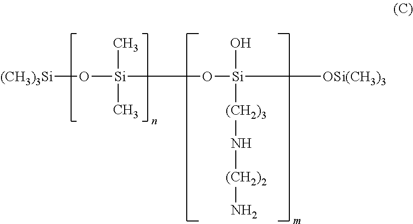

in which n and m have the meanings given above, in formula B.

Another group of amino silicone compounds corresponding to this definition is represented by silicones having the following formulae (D) or (E):

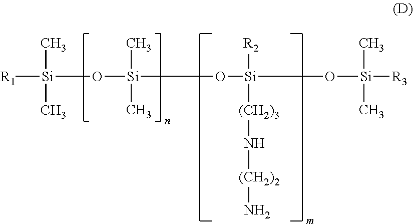

in which:

m and n are numbers such that the sum (n+m) can range from 1 to 1000, in particular from 50 to 250 and more particularly from 100 to 200, it being possible for n to denote a number from 0 to 999 and in particular from 49 to 249, and more particularly from 125 to 175, and for m to denote a number from 1 to 1000 and in particular from 1 to 10, and more particularly from 1 to 5;

R1, R2, R3, which may be identical or different, represent a hydroxy or C1-C4 alkoxy radical, where at least one of the radicals R1 to R3 denotes an alkoxy radical.

The alkoxy radical is in some embodiments a methoxy radical.

The hydroxy/alkoxy mole ratio ranges in some embodiments from 0.2:1 to 0.4:1 and in some embodiments from 0.25:1 to 0.35:1 and more particularly equals 0.3:1.

The weight-average molecular weight (Mw) of the silicone ranges in some embodiments from 2000 to 1 000 000, more particularly from 3500 to 200 000.

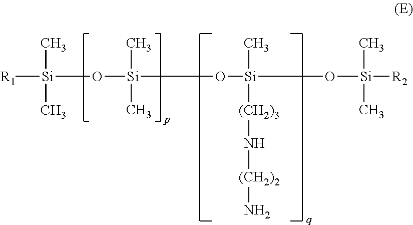

in which:

p and q are numbers such that the sum (p+q) ranges from 1 to 1000, particularly from 50 to 350, and more particularly from 150 to 250; it being possible for p to denote a number from 0 to 999 and in particular from 49 to 349, and more particularly from 159 to 239 and for q to denote a number from 1 to 1000, in particular from 1 to 10, and more particularly from 1 to 5;

R1, R2, which are different, represent a hydroxy or C1-C4 alkoxy radical, where at least one of the radicals R1 or R2 denotes an alkoxy radical.

The alkoxy radical is in some embodiments a methoxy radical.

The hydroxy/alkoxy mole ratio ranges generally from 1:0.8 to 1:1.1 and in some embodiments from 1:0.9 to 1:1 and more particularly equals 1:0.95.

The weight-average molecular weight (Mw) of the silicone ranges in some embodiments from 2000 to 200 000, even more particularly 5000 to 100 000 and more particularly from 10 000 to 50 000.

Commercial products corresponding to these silicones having structure (D) or (E) may include in their composition one or more other amino silicone compounds whose structure is different than formulae (D) or (E).

A product containing amino silicone compounds having structure (D) is sold by Wacker under the name BELSIL ADM 652.

A product containing amino silicone compounds having structure (E) is sold by Wacker under the name FLUID WR 1300@.

When these amino silicone compounds are used, one particularly advantageous embodiment consists in using them in the form of an oil-in-water emulsion. The oil-in-water emulsion may comprise one or more surfactants. The surfactants may be of any nature but are in some embodiments cationic and/or nonionic. The number-average size of the silicone particles in the emulsion generally ranges from 3 nm to 500 nanometres. In some embodiments, in particular as amino silicone compounds having formula (E), microemulsions are used whose average particle size ranges from 5 nm to 60 nanometres (limits included) and in some embodiments from 10 nm to 50 nanometres (limits included). Accordingly, according to the invention the microemulsions of amino silicone compound having formula (E) sold as FINISH CT 96 E® or SLM 28020® by Wacker can be used.

Another group of amino silicone compounds corresponding to this definition is represented by the following formula (F):

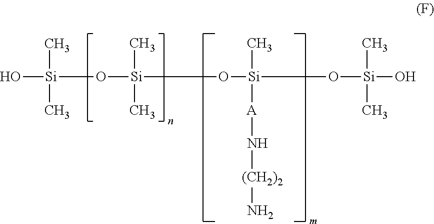

in which:

m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;

A denotes a linear or branched alkylene radical containing from 4 to 8 carbon atoms and in some embodiments 4 carbon atoms. This radical is in some embodiments linear.

The weight-average molecular weight (Mw) of these amino silicone compounds ranges in some embodiments from 2000 to 1 000 000 and even more particularly from 3500 to 200 000.

An amino silicone of formula (F) is amodimethicone (INCI name) sold under the tradename XIAMETER® MEM-8299 Cationic Emulsion by Dow Corning.

Another group of amino silicone compounds corresponding to this definition is represented by the following formula (G):

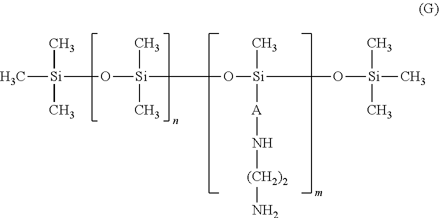

in which:

m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;

A denotes a linear or branched alkylene radical containing from 4 to 8 carbon atoms and in some embodiments 4 carbon atoms. This radical is in some embodiments branched.

The weight-average molecular weight (Mw) of these amino silicone compounds ranges in some embodiments from 500 to 1 000 000 and even more particularly from 1000 to 200 000.

A silicone having this formula is for example DC2-8566 Amino Fluid by Dow Corning.

c) amino silicone compounds corresponding to formula (H):

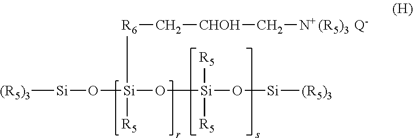

in which:

R5 represents a monovalent hydrocarbon-based radical containing from 1 to 18 carbon atoms, and in particular a C1-C18 alkyl or C2-C18 alkenyl radical, for example methyl;

R6 represents a divalent hydrocarbon-based radical, in particular a C1-C18 alkylene radical or a divalent C1-C18, for example C1-C8, alkylenoxy radical linked to the Si via an SiC bond;

Q- is an anion such as a halide ion, in particular chloride, or an organic acid salt (for example acetate);

r represents a mean statistical value from 2 to 20 and in particular from 2 to 8;

s represents a mean statistical value from 20 to 200 and in particular from 20 to 50.

Such amino silicone compounds are described more particularly in patent U.S. Pat. No. 4,185,087.

d) quaternary ammonium silicones having formula (I):

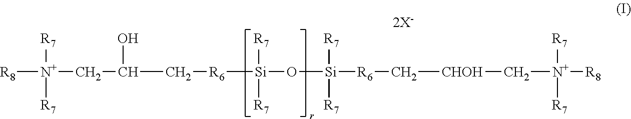

in which:

R7, which may be identical or different, represent a monovalent hydrocarbon-based radical containing from 1 to 18 carbon atoms, and in particular a C1-C18 alkyl radical, a C2-C18 alkenyl radical or a ring containing 5 or 6 carbon atoms, for example methyl;

R6 represents a divalent hydrocarbon-based radical, in particular a C1-C18 alkylene radical or a divalent C1-C18, for example C1-C8, alkylenoxy radical linked to the Si via an SiC bond;

R8, which may be identical or different, represent a hydrogen atom, a monovalent hydrocarbon-based radical containing from 1 to 18 carbon atoms, and in particular a C1-C18 alkyl radical, a C2-C18 alkenyl radical or a —R6-NHCOR7 radical;

X— is an anion such as a halide ion, in particular chloride, or an organic acid salt (for example acetate);

r represents a mean statistical value from 2 to 200 and in particular from 5 to 100;

These silicones are described, for example, in patent application EP-A 0 530 974.

e) amino silicone compounds having formula (J):

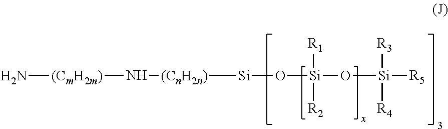

in which:

R1, R2, R3 and R4, which may be identical or different, denote a C1-C4 alkyl radical or a phenyl group;

R5 denotes a C1-C4 alkyl radical or a hydroxyl group;

n is an integer ranging from 1 to 5;

m is an integer ranging from 1 to 5;

and in which x is chosen such that the amine number is between 0.01 and 1 meq/g;

f) multiblock polyoxyalkylenated amino silicone compounds, of type (AB)n, A being a polysiloxane block and B being a polyoxyalkylenated block containing at least one amine group.

Said silicones are in some embodiments constituted of repeating units having the following general formulae:

[—(SiMe₂O)ₓSiMe₂-R—N(R″)—R′—O(C₂H₄O)ₐ(C₃H₆O)_{b}—R′—N(H)—R—]

or alternatively

[—(SiMe₂O)ₓSiMe₂-R—N(R″)—R′—O(C₂H₄O)ₐ(C₃H₆O)_{b}—]

in which:

a is an integer greater than or equal to 1, in some embodiments ranging from 5 to 200, more particularly ranging from 10 to 100;

b is an integer comprised between 0 and 200, in some embodiments ranging from 4 to 100, more particularly between from 5 and 30;

x is an integer ranging from 1 to 10 000, more particularly from 10 to 5000;

R″ is a hydrogen atom or a methyl;

R, which may be identical or different, represent a divalent linear or branched C2-C12 hydrocarbon-based radical, optionally including one or more heteroatoms such as oxygen; in some embodiments, R denotes an ethylene radical, a linear or branched propylene radical, a linear or branched butylene radical, or a —CH₂CH₂CH₂OCH(OH)CH₂— radical; preferentially R denotes a CH₂CH₂CH₂OCH(OH)CH₂— radical;

R′, which may be identical or different, represent a divalent linear or branched C2-C12 hydrocarbon-based radical, optionally including one or more heteroatoms such as oxygen; in some embodiments, R′ denotes an ethylene radical, a linear or branched propylene radical, a linear or branched butylene radical, or a —CH₂CH₂CH₂OCH(OH)CH₂— radical; preferentially R′ denotes —CH(CH₃)—CH₂—.

The siloxane blocks in some embodiments represent between 50 and 95 mol % of the total weight of the silicone, more particularly from 70 to 85 mol %.

The amine content is in some embodiments between 0.02 and 0.5 meq/g of copolymer in a 30% solution in dipropylene glycol, more particularly between 0.05 and 0.2.

The weight-average molecular weight (Mw) of the silicone is in some embodiments comprised between 5000 and 1 000 000, more particularly between 10 000 and 200 000.

Mention may be made especially of the silicones sold under the names SILSOFT A-843 or SILSOFT A+ by Momentive.

g) the alkylamino silicone compounds corresponding to formulas (K′ and K) below:

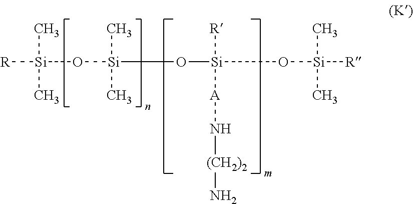

in which R, R′ and R″, which may be identical or different, each represent a C1-C4 alkyl or hydroxyl group, A represents a C3 alkylene group and m and n are such that the weight-average molecular mass of the compound is between 5000 and 500,000 approximately;

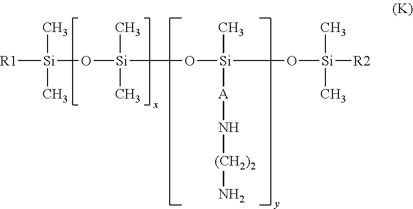

in which:

x and y are numbers ranging from 1 to 5000; in some embodiments, x ranges from 10 to 2000 and especially from 100 to 1000; in some embodiments, y ranges from 1 to 100;

R1 and R2, which may be identical or different, in some embodiments identical, are linear or branched, saturated or unsaturated alkyl radicals, comprising 6 to 30 carbon atoms, in some embodiments 8 to 24 carbon atoms and especially 12 to 20 carbon atoms;

A denotes a linear or branched alkylene radical containing from 2 to 8 carbon atoms,

In some embodiments, A comprises 3 to 6 carbon atoms, especially 4 carbon atoms; in some embodiments, A is branched. Mention may be made especially of the following divalent radicals: —CH₂CH₂CH₂ and —CH₂CH(CH₃)CH₂—.

In some embodiments, R1 and R2, which may be identical or different, are saturated linear alkyl radicals comprising 6 to 30 carbon atoms, in some embodiments 8 to 24 carbon atoms and especially 12 to 20 carbon atoms; mention may be made in particular of dodecyl, tetradecyl, pentadecyl, hexadecyl, heptadecyl, octadecyl, nonadecyl and eicosyl radicals; and preferentially, R1 and R2, which may be identical or different, are chosen from hexadecyl (cetyl) and octadecyl (stearyl) radicals.

Preferentially, the silicone is of formula (K) with:

x ranging from 10 to 2000 and especially from 100 to 1000;

y ranging from 1 to 100;

A comprising 3 to 6 carbon atoms and especially 4 carbon atoms; in some embodiments, A is branched; and more particularly A is chosen from the following divalent radicals: CH2CH2CH2 and —CH2CH(CH3)CH2-; and

R1 and R2, which may be identical or different, being linear, saturated alkyl radicals comprising 6 to 30 carbon atoms, in some embodiments 8 to 24 carbon atoms and especially 12 to 20 carbon atoms; chosen in particular from dodecyl, tetradecyl, pentadecyl, hexadecyl, heptadecyl, octadecyl, nonadecyl and eicosyl radicals; preferentially, R1 and R2, which may be identical or different, being chosen from hexadecyl (cetyl) and octadecyl (stearyl) radicals.

A silicone of formula (K) is bis-cetearyl amodimethicone (INCI name).

Mention may be made especially of the silicone sold under the name SILSOFT AX by Momentive.

The amino silicone compounds of the present disclosure may also be chosen from polydimethylsiloxanes comprising primary amine groups at the chain end or on side chains, for example aminopropyl end or side groups, for instance those of formula (A) or (B) or (C):

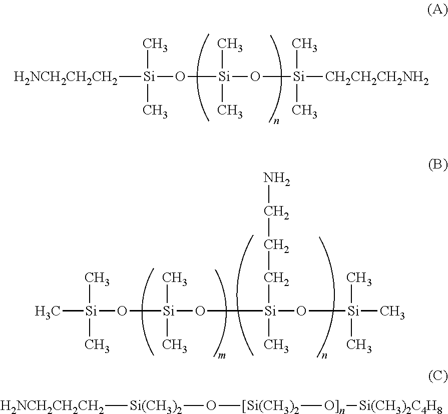

In formula (A): the value of n is such that the weight-average molecular weight of the silicone is between 500 and 55 000. As an example of aminosilicone (A), mention may be made of those sold under the names DMS-A1 1, DMS-A12, DMS-A15, DMS-A21, DMS-A31, DMS-A32 and DMS-A35 by the company Gelest.

In formula (B), the values of n and m are such that the weight-average molecular weight of the silicone is between 1000 and 55 000. As examples of silicone (B), mention may be made of those sold under the names AMS-132, AMS-152, AMS-162, AMS-163, AMS-191 and AMS-1203 by the company Gelest.

In formula (C), the value of n is such that the weight-average molecular weight of the silicone is between 500 and 3000. As an example of silicone (C), mention may be made of those sold under the names MCR-A11 and MCR-A12 by the company Gelest.

In some embodiments, the amino silicone compound according to the invention is amodimethicone (INCI name) such as the amodimethicone sold under the tradename KF 8020 from the supplier Shin Etsu or the tradename SILSOFT 253 from the supplier MOMENTIVE PERFORMANCE MATERIALS. Another some amino silicone compound is an amodimethicone of formula (F) sold under the tradename XIAMETER® MEM-8299 Cationic Emulsion by Dow Corning.

In other embodiments, the amino silicone according to the invention is chosen from bis-cetearyl amodimethicone (sold under the name SILSOFT AX by Momentive).

In other embodiments, the amino silicone according to the invention is chosen from amodimethicone.

In accordance with the various embodiments, the amino compound chosen from silicone amines is present in the compositions according to the disclosure in amounts range from about 0.1% to about 40%, and in some embodiments from about 0.1% to about 20%, and in some further embodiments from about 0.1% to about 15%. In some representative embodiments, the amino compound chosen from silicone amines is present from about 0.25% to about 10%, and from about 0.5% to about 5%. In yet other embodiments, the amino compound chosen from silicone amines is present from at least 0.5%. And in yet other embodiments, the amino compound chosen from silicone amines is present from at least 0.5% to up to about 40%. In some specific embodiments, the amino compound chosen from silicone amines is present at about 1.5%. In some other specific embodiments, the amino compound chosen from silicone amines is present at about 0.75%. In some other specific embodiments, the amino compound chosen from silicone amines compound is present at about 0.25%.

Thus, the amino compound chosen from silicone amines may be present from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 to about 40 percent by weight, including increments and ranges therein and there between.

In accordance with some particular embodiments, the silicone amines are selected from side chain amino silicones. In some examples, the silicone amines are selected from amodimethicones.

Colorants

It is known practice to dye the hair with dye compositions comprising oxidation dye precursors, which are generally known as oxidation bases. These oxidation bases are colorless or weakly colored compounds, which, when combined with oxidizing products, give rise to colored compounds via a process of oxidative condensation. It is also known that the shades obtained with these oxidation bases can be varied by combining them with couplers or coloration modifiers. The variety of molecules used as oxidation bases and couplers allows a wide range of colors to be obtained. Dye compositions comprising oxidation dye precursors are typically combined with an oxidizing composition containing an oxidizing agent; the oxidizing composition may also be called “developer composition”.

It is also known practice to dye the hair by direct dyeing. The process conventionally used in direct dyeing consists in applying to the hair direct dyes, which are colored and coloring molecules that have affinity for the hair, in leaving them to stand on the hair and then in rinsing the fibres.

The colorations resulting therefrom are particularly chromatic colorations but are, however, only temporary or semi-permanent since the nature of the interactions that bind the direct dyes to the keratin fibre and their desorption from the surface and/or the core of the fibre are responsible for their weak dyeing power and their poor fastness with respect to washing.

In certain hair coloration practices, dye compositions comprise both oxidation dye precursors and direct dyes to obtain other shades and improve the color fastness of the dyes/colorants to hair. Such dye compositions may also be combined with an oxidizing composition.

An oxidizing composition includes at least one oxidizing agent which may be chosen, for example, from peroxides, persulfates, perborates percarbonates, alkali metal bromates, ferricyanides, peroxygenated salts, or a mixture thereof. Oxidizing agents that may also be used include at least one redox enzyme such as laccases, peroxidases, and 2-electron oxidoreductases, such as uricase, where appropriate in the presence of their respective donor or co-factor. Oxygen in the air may also be employed as an oxidizing agent.

In one embodiment, the oxidizing agent is hydrogen peroxide present in an aqueous solution whose titre may range from 1 to 40 volumes, such as from 5 to 40 volumes or such as from 5 to 20 volumes.

In another embodiment, the oxidizing agent is a persulfate and/or a monopersulfate such as, for example, potassium persulfate, sodium persulfate, ammonium persulfate, as well as mixtures thereof. In one embodiment, the oxidizing agents are selected from hydrogen peroxide, potassium persulfate, sodium persulfate, and mixtures thereof.

In general, the oxidizing agent will be present in an amount of from about 0.05 to about 50% by weight, such as from about 0.1% to about 30% by weight, or such as from about 0.1% to about 20% by weight, or such as from about 1% to about 10% by weight, based on the total weight of the oxidizing composition.

The oxidizing composition can contain at least one solvent, chosen from water, organic solvents, and mixtures thereof.

Suitable organic solvents for use in the oxidizing composition include ethanol, isopropyl alcohol, propanol, benzyl alcohol, phenyl ethyl alcohol, glycols and glycol ethers, such as propylene glycol, hexylene glycol, ethylene glycol monomethyl, monoethyl or monobutyl ether, propylene glycol and its ethers, such as propylene glycol monomethyl ether, butylene glycol, dipropylene glycol, diethylene glycol alkyl ethers, such as diethylene glycol monoethyl ether and monobutyl ether, ethylene glycol, propylene glycol, butylene glycol, hexylene glycol, propane diol, glycerin, hydrocarbons such as straight chain hydrocarbons, mineral oil, polybutene, hydrogenated polyisobutene, hydrogenated polydecene, polydecene, squalane, petrolatum, isoparaffins, and mixtures, thereof.

The oxidizing composition may be in the form of a powder, gel, liquid, foam, lotion, cream, mousse, and emulsion.

Typically, a dye composition containing oxidative dye precursor can be mixed with an oxidizing composition in a ratio by weight of from about 1:0.01 to about 1:10, such as from about 1:0.01 to about 1:0.05, from about 1:0.05 to about 1:0.1, from about 1:0.1 to about 1:0.5, from about 1:0.5 to about 1:1, from about 1:1 to about 1:2, from about 1:2 to about 1:3, from about 1:3 to about 1:4, from about 1:4 to about 1:5, from about 1:5 to about 1:10. Thus, in accordance with the various embodiments, a dye composition containing oxidative dye precursor can be mixed or combined with an oxidizing composition in a ratio, by weight, from 1 to one of about 0.01, 0.02, 0.03, 0.04, 0.05, 0.06, 0.07, 0.08, 0.09, 0.1, 0.2, 03, 0.4, 0.5, 0.6, 0.7, 0.8, 0.9, 1, 2, 3, 4, 5, 6, 7, 8, 9, and 10, including increments and ranges therein and there between from about 1:0.01 to about 1:10.

Solvent

The composition, according to the present disclosure, further includes suitable solvents for treatment of keratinous fibers. Examples of suitable solvents include water, in some embodiments, distilled or de-ionised, or organic solvents as carriers and solvents for the polycarbodiimides and amino compounds chosen from polyamines and silicone amines, and optional colorants or dyes, and mixtures thereof.

Suitable organic solvents may be chosen from volatile and nonvolatile organic solvents.

Suitable organic solvents are typically C1-C4 lower alcohols, glycols, polyols, polyol ethers, hydrocarbons, and oils. Examples of organic solvents include, but are not limited to, ethanol, isopropyl alcohol, benzyl alcohol, phenyl ethyl alcohol, isododecane, propylene glycol, pentylene glycol, hexylene glycol, glycerol, and mixtures thereof.

Other suitable organic solvents include glycol ethers, for example, ethylene glycol and its ethers such as ethylene glycol monomethyl ether, ethylene glycol monopropyl ether, ethylene glycol monobutyl ether, propylene glycol and its ethers, such as propylene glycol monomethyl ether, propylene glycol monopropyl ether, propylene glycol monobutyl ether, dipropylene glycol and diethylene glycol alkyl ethers, such as diethylene glycol monoethyl ether, diethylene glycolmonobutyl ether, and dipropylene glycol n-butyl ether. Glycol ethers are commercially available from The Dow Chemical Company under the DOW E-series and DOW P-series. An exemplary glycol ether for use in the present invention is dipropylene glycol n-butyl ether, known under the tradename of DOWANOL DPnB.

Suitable organic solvents also include synthetic oils and hydrocarbon oils include mineral oil, petrolatum, and C10-C40 hydrocarbons which may be aliphatic (with a straight, branched or cyclic chain), aromatic, arylaliphatic such as paraffins, iso-paraffins, isododecanes, aromatic hydrocarbons, polybutene, hydrogenated polyisobutene, hydrogenated polydecene, polydecene, squalene, petrolatum and isoparaffins, silicone oils, fluoro oils and mixtures, thereof.

The term “hydrocarbon based oil” or “hydrocarbon oil” refers to oil mainly containing hydrogen and carbon atoms and possibly oxygen, nitrogen, sulfur and/or phosphorus atoms. Representative examples of hydrocarbon based oils include oils containing from 8 to 16 carbon atoms, and especially branched C8 C16 alkanes (also known as isoparaffins), for instance isododecane (also known as 2,2,4,4,6 pentamethylheptane), isodecane and isohexadecane.

Examples of silicone oils that may be useful in the present invention include nonvolatile silicone oils such as polydimethylsiloxanes (PDMS), polydimethylsiloxanes comprising alkyl or alkoxy groups that are pendent and/or at the end of a silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenyl methyldiphenyl trisiloxanes and 2 phenylethyl trimethylsiloxysilicates, and dimethicones or phenyltrimethicones with a viscosity of less than or equal to 100 cSt.

Other representative examples of silicone oils that may be useful in the present invention include volatile silicone oils such as linear or cyclic silicone oils, and especially containing from 2 to 10 silicon atoms and in particular from 2 to 7 silicon atoms, these silicones optionally comprising alkyl or alkoxy groups containing from 1 to 10 carbon atoms. Specific examples include dimethicones with a viscosity of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Representative examples of fluoro oils that may be suitable for use in the present invention include volatile fluoro oils such as nonafluoromethoxybutane and perfluoro methylcyclopentane.

Particularly suitable solvents in a composition of the present disclosure include water, isododecane, ethanol, and combinations thereof. The solvent will typically be present in total amounts ranging from about 60% to 98%, in some embodiments, from 80% to 96%, by weight, including all ranges and subranges therebetween, based on the total weight of the composition. Thus, solvents may be present in amounts of about 98%, 97%, 96%, 95%, 94%, 93%, 92%, 91%, 90%, 85%, 80%, 75%, 70%, 65%, 60%, and about 55%, 50%, 48%, 46%, 45%, 44%, 42%, 40%, by weight, based on the total weight of the composition, including increments and ranges therein and there between from about 40% to about 98%.

In yet some other embodiments, the solvent of the present disclosure does not comprise water and/or organic solvent that is added as a separate ingredient, by itself, into the compositions of the present invention, such that water and/or organic solvent is present in the compositions of the present invention when it accompanies one or more ingredients of a raw material that is added into the compositions of the invention.

When the compositions of the disclosure contain water, according to various embodiments, water can be present in amounts of about 98% or less, such as about 96%, 95%, 90%, 85%, 80%, 75%, 70%, 65%, 60%, 55%, 50%, 48%, 46%, 45%, 44%, 42%, 40%, 35%, 30%, 20%, 10%, or 5% or less, by weight, based on the total weight of the composition, including increments and ranges therein and there between.

When the compositions of the disclosure contain an organic solvent(s), according to various embodiments, the organic solvent(s) can be present in a total amount of about 98% or less, such as about 96%, 95%, 90%, 85%, 80%, 75%, 70%, 65%, 60%, 55%, 50%, 48%, 46%, 45%, 44%, 42%, 40%, 35%, 30%, 20%, 10%, or 5% or less, by weight, based on the total weight of the composition, including increments and ranges therein and there between.

Additives

The composition, according to the present disclosure, further includes suitable additives for treatment of keratinous fibers.

The composition according to the disclosure may also comprise additives chosen from amino compounds other than the amino compounds comprising polyvinylamines of the present invention, for example any of the other amino compounds as listed herein, and amino silicones and amino functionalized silane compounds, surfactants (anionic, nonionic, cationic and amphoteric/zwitterionic), and polymers other than the polycarbodiimide compounds of the invention such as anionic polymers, nonionic polymers, amphoteric polymers, polymeric rheology modifiers, thickening and/or viscosity modifying agents, associative or non-associative polymeric thickeners. Other suitable additives may be chosen from non-polymeric thickeners, nacreous agents, opacifiers, dyes or pigments, fragrances, mineral, plant or synthetic oils, waxes including ceramides, vitamins, UV-screening agents, free-radical scavengers, antidandruff agents, hair-loss counteractants, hair restorers, preserving agents, pH stabilizers and solvents, and mixtures thereof.

The compositions of certain embodiments may comprise stabilizers, for example sodium chloride, magnesium dichloride or magnesium sulfate.

The amino silicones that may be employed in the compositions of the present disclosure may be chosen from polysiloxanes having at least one primary, secondary or tertiary amine group such as trimethylsilylamodimethicones, quaternary ammonium silicones, multiblock polyoxyalkylenated amino silicones, of type (AB)n, A being a polysiloxane block and B being a polyoxyalkylenated block containing at least one amine group, alkyl amino silicones, and mixtures thereof.

Suitable examples of the amine or amino silicone compounds include amodimethicone (e.g., sold under the name KF 8020 by Shin Etsu or XIAMETER® MEM-8299 Cationic Emulsion by Dow Corning), and bis-cetearyl amodimethicone (sold under the name SILSOFT AX by Momentive).

The rheology modifiers and thickening/viscosity-modifying agents that may be employed in compositions of the present disclosure may include any water-soluble or water-dispersible compound that is compatible with the polycarbodiimide, amino compounds, and compositions of the disclosure, such as acrylic polymers (in particular, Acrylates/C10-30 Alkyl Acrylate Crosspolymer, carbomers, acrylate copolymers, acrylate crosspolymers), non-acrylic polymers, starch, saccharide-based polymers (e.g., guar, guar gums), cellulose-based polymers (in particular, hydroxyethylcellulose, cellulose gums, alkyl hydroxyethyl cellulose), non-polymeric and polymeric gelling agents, silica particles, clay, and mixtures thereof.

Additional Optional Additives

Polycarboxylic-Acid Polymer Compounds

The polycarboxylic acid, compounds of the present disclosure may be chosen from anionic, nonionic, and amphoteric polymers.

The anionic polymers may be soluble in a cosmetically acceptable medium or insoluble in this same medium such that they may be used in the form of dispersions of solid or liquid particles of polymer (latex or pseudolatex).

The anionic polymers may be selected from polymers comprising groups derived from carboxylic acids, and have an average molecular weight by number of between about 500 and 5,000,000. The carboxylic groups are provided by unsaturated mono- or diacid carboxylic monomers such as those that have the formula:

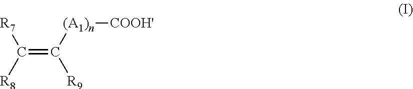

in which n is an integer from 0 to 10, A₁ denotes a methylene group optionally joined to the carbon atom of the unsaturated group or to the adjacent methylene group when n is greater than 1, via a heteroatom such as oxygen or sulfur, R₇ denotes a hydrogen atom or a phenyl or benzyl group, R₈ denotes a hydrogen atom or a lower alkyl or carboxyl group, and R₉ denotes a hydrogen atom, a lower alkyl group, a CH₂—COOH, phenyl or benzyl group.

In the abovementioned formula, a lower alkyl group in some embodiments denotes a group containing 1 to 4 carbon atoms and in particular methyl and ethyl groups. Examples of anionic polymers containing carboxyl groups in accordance with the invention are:

The polycarboxylic acid compounds include Copolymers of acrylic or methacrylic acid or salts thereof, and in particular copolymers of acrylic acid and acrylamide sold in the form of their sodium salts;

The polycarboxylic acid compounds include Copolymers of acrylic or methacrylic acid with a monoethylenic monomer such as ethylene, styrene, vinyl esters and acrylic or methacrylic acid esters, optionally grafted onto a polyalkylene glycol such as polyethylene glycol and optionally crosslinked. The polycarboxylic acid compounds include methacrylic acid/acrylic acid/ethyl acrylate/methyl methacrylate copolymers in an aqueous dispersion.

The polycarboxylic acid compounds include Crotonic acid copolymers, such as those comprising vinyl acetate or propionate units in their chain and optionally other monomers such as allyl esters or methallyl esters, vinyl ether or vinyl ester of a linear or branched saturated carboxylic acid with a long hydrocarbon-based chain, such as those containing at least 5 carbon atoms, it being possible for these polymers optionally to be grafted or crosslinked, or alternatively another vinyl, allyl or methallyl ester monomer of an [alpha]- or [beta]-cyclic carboxylic acid.

The polycarboxylic acid compounds include Copolymers of C4-C8 monounsaturated carboxylic acids selected from: copolymers comprising (i) one or more maleic, fumaric, itaconic, allyloxyacetic, methallyloxyacetic, 3-allyloxypropionic, allylthioacetic, allylaminoacetic, vinylacetic, vinyloxyacetic, crotyloxyacetic, 3-butenoic, 4-pentenoic, 10-undecenoic, allylmalonic, maleamic, itaconamic, N-monohydroxyalkyl- or N-dihydroxy-alkyl-maleamic acids and (ii) at least one monomer selected from vinyl esters, vinyl ethers, vinyl halides, phenylvinyl derivatives, acrylic acid and its esters, the anhydride functions of these copolymers optionally being monoesterified or monoamidated. The polycarboxylic acid compounds include copolymers comprising (i) one or more maleic, citraconic or itaconic anhydride units and (ii) one or more monomers selected from allyl or methallyl esters optionally comprising one or more acrylamide, methacrylamide, [alpha]-olefin, acrylic or methacrylic ester, acrylic or methacrylic acid or vinylpyrrolidone groups in their chain, the anhydride functions of these copolymers optionally being monoesterified or monoamidated. The polycarboxylic acid compounds include polyacrylamides comprising carboxylate groups.

The polycarboxylic acid compounds of the present disclosure may also include those anionic polymers as sold under the FIXATE series as commercially available from Lubrizol, such as a branched block anionic polymer sold as FIXATE G-100, a branched anionic acrylate copolymer Polyacrylate-2 Crosspolymer (FIXATE SUPERHOLD polymer), Acrylates Crosspolymer-3 (FIXATE FREESTYLE Polymer), Polyacrylate-14 (FIXATE PLUS Polymer), those sold under the CARBOPOL series as commercially available from Lubrizol such as Acrylates Crosspolymer-4 (CARBOPOL AQUA SF-2), Acrylates Crosspolymer-4 (CARBOPOL AQUA CC), and those sold under the SYNTRAN series as commercially available from Interpolymer such as Acrylates Copolymer (SYNTRAN 5190), Styrene/Acrylates/Ammonium Methacrylate Copolymer (SYNTRAN 5760), and Ammonium Acrylates Copolymer (SYNTRAN KL-219C).

The polycarboxylic acid compounds of the present disclosure also includes anionic latex polymers such as acrylic copolymer and (meth)acrylate copolymers dispersions.

The polycarboxylic acid compounds include copolymers of acrylic acid or of acrylic esters, such as Acrylates/t-Butylacrylamide copolymer sold as ULTRAHOLD 8, acrylic acid/ethyl acrylate/N-tert-butylacrylamide terpolymers sold especially as ULTRAHOLD STRONG by BASF, copolymers derived from crotonic acid, such as vinyl acetate/vinyl tert-butylbenzoate/crotonic acid terpolymers and the crotonic acid/vinyl acetate/vinyl neododecanoate terpolymers sold especially as RESYN 28-29-30 by Azko Nobel, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold, for example, as GANTREZ AN or ES by ISP, the copolymers of methacrylic acid and methyl methacrylate sold as EUDRAGIT L by Rohm Pharma, the copolymers of methacrylic acid and ethyl acrylate sold as LUVIMER MAEX or MAE by BASF, the vinyl acetate/crotonic acid copolymers sold as LUVISET CA 66 by BASF, the vinyl acetate/crotonic acid copolymers grafted with polyethylene glycol sold as ARISTOFLEX A by BASF, and the polymer sold as FIXATE G-100 by Noveon.

The polycarboxylic acid compounds include amphoteric polymers which may be selected from the following polymers:

copolymers having acidic vinyl units and basic vinyl units, such as those resulting from the copolymerization of a monomer derived from a vinyl compound bearing a carboxylic group such as, more particularly, acrylic acid, methacrylic acid, maleic acid, alpha-chloroacrylic acid, and a basic monomer derived from a substituted vinyl compound containing at least one basic atom, such as, more particularly, dialkylaminoalkyl methacrylate and acrylate, dialkylaminoalkylmethacrylamides and acrylamides. Such compounds are described in U.S. Pat. No. 3,836,537.

The polycarboxylic acid compounds include Polymers comprising units derived from:

at least one monomer selected from acrylamides and methacrylamides substituted on the nitrogen atom with an alkyl group,

at least one acidic comonomer containing one or more reactive carboxylic groups, selected more particularly from acrylic, methacrylic, crotonic, itaconic, maleic and fumaric acid and alkyl monoesters, having 1 to 4 carbon atoms, of maleic or fumaric acid or anhydride, and

at least one basic comonomer such as esters with primary, secondary, tertiary or quaternary amine substituents of acrylic and methacrylic acids and the product of quaternization of dimethylaminoethyl methacrylate with dimethyl or diethyl sulfate. The N-substituted acrylamides or methacrylamides that are more some according to the invention are compounds in which the alkyl groups contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides. Some basic comonomers are aminoethyl, butylaminoethyl, N,N′-dimethylaminoethyl and N-tert-butylaminoethyl methacrylates.

The copolymers whose CTFA (4th edition, 1991) name is octylacrylamide/acrylates/butylaminoethyl methacrylate copolymer, such as the products sold as AMPHOMER LV 71, Acrylates/octylacrylamide copolymer sold as Amphomer 28-4961 or LOVOCRYL 47 by National Starch, are particularly used.

The polycarboxylic acid compounds include Crosslinked and acylated polyaminoamides.

The polycarboxylic acid compounds include carboxylic acids selected from acids having 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid and itaconic acid.

The polycarboxylic acid compounds include Polymers comprising zwitterionic units of formula:

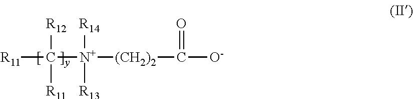

in which R11 denotes a polymerizable unsaturated group such as an acrylate, methacrylate, acrylamide or methacrylamide group, y and z represent an integer from 1 to 3, R12 and R13 represent a hydrogen atom, a methyl, ethyl or propyl group, R14 and R15 represent a hydrogen atom or an alkyl group such that the sum of the carbon atoms in R14 and R15 does not exceed 10.

The polymers comprising such units may also comprise units derived from non-zwitterionic monomers such as dimethyl- or diethylaminoethyl acrylate or methacrylate or alkyl acrylates or methacrylates, acrylamides or methacrylamides or vinyl acetate, for example, methyl methacrylate/methyl dimethylcarboxymethylammonioethyl methacrylate copolymers such sold as DIAFORMER Z301 by Sandoz; The polycarboxylic acid compounds include Polymers derived from chitosan comprising monomer units corresponding to the following formulae:

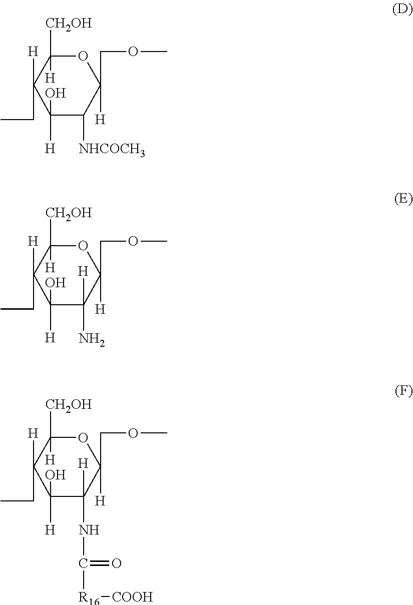

the unit (D) being present in proportions of between 0 and 30 percent, the unit (E) in proportions of between 5 percent and 50 percent and the unit (F) in proportions of between 30 percent and 90 percent, it being understood that, in this unit (F), R16 represents a group of formula:

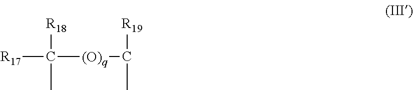

in which, if q=0, R17, R18 and R19, which may be identical or different, each represent a hydrogen atom, a methyl, hydroxyl, acetoxy or amino residue, a monoalkylamine residue or a dialkylamine residue that are optionally interspersed with one or more nitrogen atoms and/or optionally substituted with one or more amine, hydroxyl, carboxyl, alkylthio or sulfonic groups, an alkylthio residue in which the alkyl group bears an amino residue, at least one of the groups R17, R18 and R19 being, in this case, a hydrogen atom;

or, if q=1, R17, R18 and R19 each represent a hydrogen atom, and also the salts formed by these compounds with bases or acids.

The polycarboxylic acid compounds include Polymers with units corresponding to the general formula below are described, for example, in French patent 1 400 366:

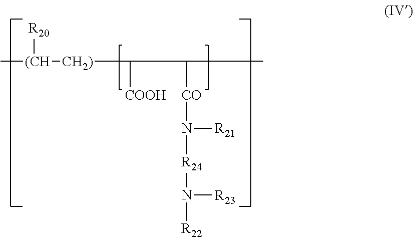

in which R20 represents a hydrogen atom, a CH3O, CH3CH2O or phenyl group, R21 denotes a hydrogen atom or a lower alkyl group such as methyl or ethyl, R22 denotes a hydrogen atom or a C1-C6 lower alkyl group such as methyl or ethyl, R23 denotes a C1-C6 lower alkyl group such as methyl or ethyl or a group corresponding to the formula: —R24-N(R22)2, R24 representing a group —CH2-CH2-, —CH2-CH2-CH2- or —CH2-CH(CH3)-, R22 having the meanings mentioned above.

The polycarboxylic acid compounds include Polymers derived from the N-carboxyalkylation of chitosan, such as N-carboxymethylchitosan or N-carboxybutylchitosan.

The polycarboxylic acid compounds include Amphoteric polymers of the type -D-X-D-X selected from:

Polymers obtained by the action of chloroacetic acid or sodium chloroacetate on compounds comprising at least one unit of formula:

-D-X-D-X-D- (V′) where D denotes a

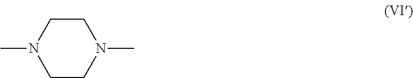

group and X denotes the symbol E or E′; E or E′ may be identical or different and denote a divalent group that is an alkylene group with a straight or branched chain containing up to 7 carbon atoms in the main chain, which is unsubstituted or substituted by hydroxyl groups and which may comprise, in addition to oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine or alkenylamine groups, hydroxyl, benzylamine, amine oxide, quaternary ammonium, amide, imide, alcohol, ester and/or urethane groups.

Polymers having the formula:

-D-X-D-X— (VI′) where D denotes a

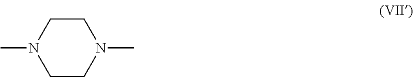

group and X denotes the symbol E or E′ and at least once E′; E having the meaning given above and E′ is a divalent group that is an alkylene group with a straight or branched chain having up to 7 carbon atoms in the main chain, which is unsubstituted or substituted with one or more hydroxyl groups and containing one or more nitrogen atoms, the nitrogen atom being substituted with an alkyl chain that is optionally interspersed by an oxygen atom and necessarily comprising one or more carboxyl functions or one or more hydroxyl functions and betainized by reaction with chloroacetic acid or sodium chloroacetate.

The polycarboxylic acid compounds include (C1-C5) Alkyl vinyl ether/maleic anhydride copolymers partially modified by semiamidation with an N,N-dialkylaminoalkylamine such as N,N-dimethylaminopropylamine or by semiesterification with an N,N-dialkylaminoalkanol. These copolymers may also comprise other vinyl comonomers such as vinylcaprolactam.

Among the amphoteric polymers described above, the ones that are most some are Octylacrylamide/acrylates/butylamino ethyl methacrylate copolymer, such as the products sold as AMPHOMER, AMPHOMER LV 71 or LOVOCRYL 47 by National Starch and the copolymers of methyl methacrylate/methyl dimethylcarboxy-methylammonioethyl methacrylate, sold, for example, as DIAFORMER Z301 by Sandoz.

The polycarboxylic acids of the present disclosure may be chosen from compounds which are known to be used as rheology modifiers or thickeners in cosmetic compositions. Such polymers include anionic and amphoteric polymers, for example crosslinked homopolymers of acrylic acid, associative polymers, non-associative thickening polymers, and water-soluble thickening polymers. Such polymers may also be chosen from nonionic, anionic, cationic and amphoteric amphiphilic polymers. The rheology modifiers or thickeners that can used may include those polycarboxylic acid compounds described above.

The amphiphilic polymers may, optionally, contain a hydrophobic chain that is a saturated or unsaturated, aromatic or non-aromatic, linear or branched C6-C30 hydrocarbon-based chain, optionally comprising one or more oxyalkylene (oxyethylene and/or oxypropylene) units.

Representative examples of such amphiphilic polymers are:

nonionic amphiphilic polymers containing a hydrophobic chain such as:

copolymers of C1-C6 alkyl(meth)acrylates and of amphiphilic monomers containing a hydrophobic chain;

copolymers of hydrophilic (meth)acrylates and of hydrophobic monomers containing at least one hydrophobic chain, for instance the polyethylene glycol methacrylate/lauryl methacrylate copolymer;

anionic amphiphilic polymers containing at least one hydrophobic chain which are crosslinked or non-crosslinked, contain at least one hydrophilic unit derived from one or more ethylenically unsaturated monomers bearing a carboxylic acid function, which is free or partially or totally neutralized, and at least one hydrophobic unit derived from one or more ethylenically unsaturated monomers bearing a hydrophobic side chain, and optionally at least one crosslinking unit derived from one or more polyunsaturated monomers.

Examples of anionic amphiphilic polymers include CARBOPOL ETD-2020 (acrylic acid/C10-C30 alkyl methacrylate crosslinked copolymer sold by the company Noveon); CARBOPOL 1382, PEMULEN TR1 and PEMULEN TR2 (acrylic acid/C10-C30 alkyl acrylatecrosslinked copolymers-sold by the company Noveon), the methacrylic acid/ethyl acrylate/oxyethylenated stearyl methacrylate copolymer (55/35/10); the (meth)acrylic acid/ethyl acrylate/25 EO oxyethylenated behenyl methacrylate copolymer (ACULYN 28 sold by Rohm and Haas) and the methacrylic acid/ethyl acrylate/steareth-10 allyl ether crosslinked copolymer.

Other examples include cross-linked acrylic polymers, for example those sold under the CARBOPOL SF series, such as ethyl acrylate/methacrylic acid copolymer with INCI name: acrylates copolymer, sold under the name CARBOPOL SF1(R) by the LUBRIZOL company.

Yet other examples include anionic polymers also known as anionic thickening polymers chosen from carbomers, acrylate copolymers, and crosslinked terpolymers of methacrylic acid, ethylacrylate, and polyethylene glycol (10 EO) stearyl alcohol ether (Steareth 10), such as the products sold by the company ALLIED COLLOIDS under the names SALCARE SC 80 and SALCARE SC 90, which are aqueous emulsions containing 30 percent of a crosslinked terpolymer of methacrylic acid, of ethyl acrylate and of steareth-10-allyl ether (40/50/10).

Anionic thickening polymers can also be chosen from:

terpolymers formed from maleic anhydride/C30-C38 alpha-olefin/alkyl maleate such as the product (maleic anhydride/C30-C38 alpha-olefin/isopropyl maleate copolymer) sold under the name PERFORMA 1608 by the company NEWPHASE TECHNOLOGIES;

acrylic terpolymers formed from: (a) 20 percent to 70 percent by weight of a carboxylic acid with alpha, beta-monoethylenic unsaturation; (b) 20 percent to 80 percent by weight of a nonsurfactant monomer with alpha, beta-monoethylenic unsaturation different from (a); and (c) 0.5 percent to 60 percent by weight of a nonionic monourethane which is the product of the reaction of a monohydric surfactant with a monoisocyanate with monoethylenic unsaturation; (3) copolymers formed from at least two monomers, wherein at least one of the two monomers is chosen from a carboxylic acid with alpha, beta-monoethylenic unsaturation, an ester of a carboxylic acid with alpha, beta-monoethylenic unsaturation, and an oxyalkylenated fatty alcohol; and (4) copolymers formed from at least three monomers, wherein at least one of the three monomers is chosen from a carboxylic acid with alpha, beta-monoethylenic unsaturation, at least one of the three monomers is chosen from an ester of a carboxylic acid with alpha beta-monoethylenic unsaturation and at least one of the three monomers is chosen from an oxyalkylenated fatty alcohol.

Additionally, these compounds can also contain, as a monomer, a carboxylic acid ester comprising an alpha, beta-monoethylenic unsaturation and a C1-C4 alcohol. By way of example of this type of compound, there may be mentioned ACULYN 22 sold by the company ROHM and HAAS, which is an oxyalkylenated stearyl methacrylate/ethylacrylate/methacrylic acid terpolymer.

The polycarboxylic acid compounds include associative polyurethanes, associative unsaturated polyacids, and associative polymers or copolymers containing at least one monomer comprising ethylenic unsaturation.

A representative example of an associative polyurethane is methacrylic acid/methyl acrylate/ethoxylated (40 EO) behenyl alcohol dimethyl(meta-isopropenyl)benzyl isocyanate terpolymer as a 25 percent aqueous dispersion, known by the trade name, VISCOPHOBE DB 1000 and commercially available from Amerchol.

According to some embodiments of the present invention, the polycarboxylic acid compounds include at least one acrylic acid-based, (meth)acrylic acid-based, acrylate-based or (meth)acrylate-based monomer having anionic and/or cationic functionalities. Suitable compounds include, but are not limited to, polymers comprising polyacrylates such as those identified in the International Cosmetic Ingredient Dictionary and Handbook (9 th ed. 2002) such as, for example, polyacrylate-1, polyacrylate-2, polyacrylate-3, polyacrylate-4, polyacrylate-16, polyacrylate-17, polyacrylate-18, polyacrylate-19, polyacrylate-21, and mixtures thereof. Such (co)polymers, or similar (co)polymers, can be combined individually or with other (co)polymers in such a way to form suitable bimodal agents having both cationic and anionic functionalities. According to certain embodiments, the bimodal agent is selected from the group consisting of polymers consisting of polyacrylate-21 and acrylates/dimethylaminoethylmethacrylate copolymer (marketed under the name SYNTRAN PC 5100 by Interpolymer), polyacrylate-16 (marketed under the name SYNTRAN PC 5112 by Interpolymer), and polyacrylate-18 and polyacrylate-19 (marketed under the names SYNTRAN PC 5107 or SYNTRAN PC 5117 by Interpolymer).

Silicone Polymers Having at Least One Carboxylic Acid Group (Carboxysilicone Polymers).

The silicone polymers having at least one carboxylic acid group, referred herein as carboxysilicone polymers, according to the present disclosure, may be an organopolysiloxane comprising:

(A) a compound having the following formula:

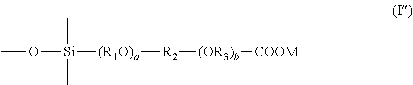

wherein R₁ and R₃ independently denote a linear or branched alkylene radical containing from 2 to 20 carbon atoms and R₂ denotes a linear or branched alkylene radical containing from 1 to 50 carbon atoms which can comprise a hydroxyl group, a represents 0 or 1, b is a number ranging from 0 to 200 and M denotes hydrogen, an alkali metal or alkaline-earth metal, NH₄ or a quaternary ammonium group, such as a mono-, di-, tri- or tetra(C1-C4 alkylammonium) group, R₁ and R₃ can denote, for example, ethylene, propylene or butylene, or

(B) a group comprising at least one pyrrolidone carboxylic acid unit having the following formula:

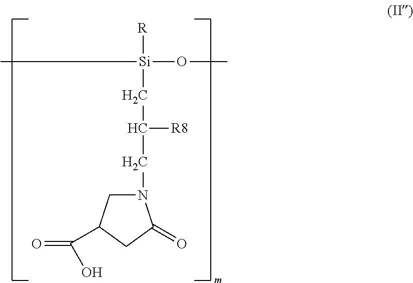

in which R is selected from methyl or phenyl; R8 is hydrogen or methyl, m is an integer from 1 to 1000, or
(C) a group comprising at least one polyvinyl acid/ester unit (C) resulting from the polymerization of Divinyl-PDMS, Crotonic Acid, Vinylacetate, and Vinyl Isoalkylester,

and combinations of (A), (B) and (C).

Suitable carboxysilicone polymers include, for example, a silicone polymer comprising at least one carboxylic acid group chosen from organopolysiloxanes of formula:

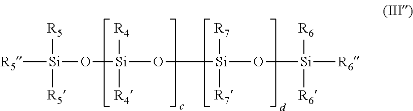

wherein the radicals R4, R4′ are identical to or different from each other and are chosen from a linear or branched C1-C22 alkyl radical, a C1-C22 alkoxy radical and a phenyl radical, the radicals R5, R5′, R5″, R6, R6′, R6″, R7, and R7′ are identical to or different from each other and are chosen from a linear or branched C1-C22 alkyl radical, a C1-C22 alkoxy radical, a phenyl radical, a radical —(R10O)a-R2-(OR3)b-COOM, a radical containing pyrrolidone carboxylic acid, a radical of polyvinyl acid/ester; and

wherein at least one of the radicals R5, R6 and R7 is a radical chosen from a radical —(R10O)a-R2-(OR3)b-COOM, a radical containing pyrrolidone carboxylic acid, a radical of polyvinyl acid/ester;

wherein R1, R2, R3, a, b and M have the same meaning as described in Unit (A) above;

wherein c and d are integers from 0 to 1000, the sum c+d in some particular embodiments ranging from 1 to 1000 or from 2 to 1000.

Among the carboxysilicone polymers of formula (III″) that comprise at least one unit (I″), which in some particular embodiments are the compounds of formula below:

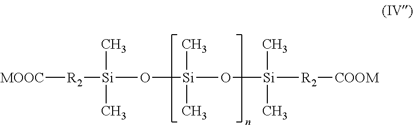

wherein R2, and M have the same meaning as described in Unit (A) above, n is an integer from 1 to 1000. Examples are: dual-end carboxy silicones X-22-162C from Shin Etsu and Silform INX (INCI name: Bis-Carboxydecyl Dimethicone) from Momentive.

Other exemplary embodiments organopolysiloxanes of formula (III″) are the ones of formula:

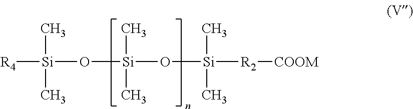

in which R₂, R₄, n, and M having the same meaning as in Unit (A) above. An example is a single-end carboxy silicone X-22-3710 from Shin Etsu.

Other exemplary embodiments organopolysiloxanes of formula (III″) are the ones of formula:

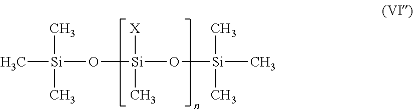

wherein X is a radical —(R10O)a-R2-(OR3)b-COOM wherein R1, R2, R3, a, b and M have the same meaning as described in Unit (A) above.

Even more particularly, the compounds of formula (VI′) in which a and b are equal to 0 and R₂ is a linear or branched C₂-C₁₂ alkylene group such as (CH₂)₉, (CH₂)₁₀ or —CH(CH₃)— are exemplary embodiments. An example is a side-chain carboxy silicone X-22-3701 E from Shin Etsu.

Among the organopolysiloxanes of formula (III″) that contain unit (B), exemplary embodiments include the compounds of formula below:

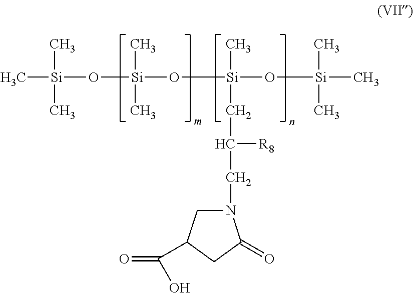

wherein R8, m, are defined as in Unit (B) above and n is an integer from 1 to 1000. An example is Grandsil PCA such as in Grandsil SiW-PCA-10 (INCI name: Dimethicone (and) PCA Dimethicone (and) Butylene Glycol (and) Decyl Glucoside from Grant Industries.

Among the organopolysiloxanes of formula (III″) that contain polyvinyl acid/ester Unit (C), exemplary embodiments are crosslinked anionic copolymers comprised of organic polymer blocks and silicone blocks, resulting in a multiblock polymer structure. In particular, the silicone-organic polymer compound of the present invention may be chosen from crosslinked anionic copolymers comprising at least one crosslinked polysiloxane structural unit. An example of such a branched multi-block carboxysilicone polymer is Belsil® P1101 (may also be known under the tradename Belsil® P1101) (INCI name: Crotonic Acid/Vinyl C8-12 Isoalkyl Esters/VA/Bis-Vinyldimethicone Crosspolymer, also known by the technical name of Crotonic Acid/Vinyl C8-12 Isoalkyl Esters/VA/divinyldimethicone Crosspolymer) from Wacker Chemie AG.

Additional suitable carboxysilicone polymers are described, for example, in patent applications WO 95/23579 and EP-A-0,219,830, which are hereby incorporated by reference in their entirety.

Compounds corresponding to formula (VI″) above are sold, for example, under the name HUILE M 642 by the company Wacker, under the names SLM 23 000/1 and SLM 23 000/2 by the company Wacker, under the name 176-12057 by the company General Electric, under the name FZ 3703 by the company OSI and under the name BY 16 880 by the company Toray Silicone.

Other non-limiting examples of carboxysilicone polymers are silicone carboxylate containing polymers (silicone carboxylates).

Suitable silicone carboxylates may be chosen from water soluble silicone compounds comprising at least one carboxylic acid group, oil soluble silicone compounds comprising at least one carboxylic acid group, water-dispersible silicone compounds comprising at least one carboxylic acid group, and silicone compounds comprising at least one carboxylic acid group which are soluble in organic solvents. In one embodiment, the silicone carboxylate further comprises at least one alkoxylated chain, wherein the at least one alkoxy group may be chosen from terminal alkoxy groups, pendant alkoxy groups, and alkoxy groups which are intercalated in the skeleton of the at least one silicone compound. Non-limiting examples of at least one alkoxy group include ethylene oxide groups and propylene oxide groups.

The at least one carboxylic acid group may be chosen from terminal carboxylic acid groups and pendant carboxylic acid groups. Further, the at least one carboxylic acid may be chosen from carboxylic acid groups in free acid form, i.e., —COOH, and carboxylic acid groups in salt form, i.e.,—COOM, wherein M may be chosen from inorganic cations, such as, for example, potassium cations and sodium cations, and organic cations.

In one embodiment, the silicone carboxylate is a compound of formula:

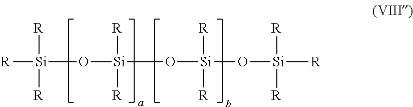

wherein a is an integer ranging from 1 to 100; b is an integer ranging from 0 to 500; and R, which may be identical or different, are each chosen from optionally substituted hydrocarbon groups comprising from 1 to 9 carbon atoms, optionally substituted phenyl groups, and groups of the following formula:

—(CH₂)₃—O-(EO)_{c}—(PO)_{d}-(EO)ₑ—C(O)—R′—C(O)—OH (IX″)

wherein c, d, and e, which may be identical or different, are each integers ranging from 0 to 20; EO is an ethylene oxide group; PO is a propylene oxide group; and R′ is chosen from optionally substituted divalent hydrocarbons, such as alkylene groups and alkenylene groups comprising from 2 to 22 carbon atoms, and optionally substituted divalent aromatic groups, such as groups of the following formula:

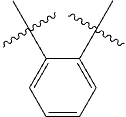 [SMILES: *c1ccccc1*]

and groups of the following formula:

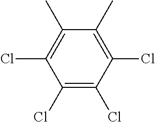

with the proviso that at least one of the R groups is chosen from groups of formula (XIV) and with the further proviso that when only one of the R groups is chosen from groups of formula (XIV), the other R groups are not all methyl groups.

Non-limiting examples of silicone carboxylates include those commercially available from Noveon under the name Ultrasil® CA-1 Silicone (Dimethicone PEG-7 Phthalate) and Ultrasil® CA-2 Silicone (Dimethicone PEG-7 Succinate), both of which correspond to formula (XV) below. Thus, in one embodiment, the at least one silicone carboxylate is chosen from a compound of formula below and salts thereof:

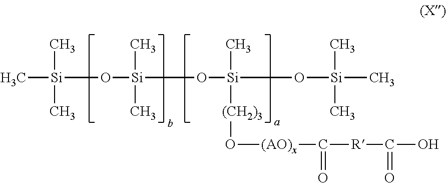

wherein a is an integer ranging from 1 to 100, b is an integer ranging from 0 to 500, AO is chosen from groups of the following formula:

-(EO)_{c}—(PO)_{d}-(EO)ₑ—

wherein c, d, and e, which may be identical or different, are each integers ranging from 0 to 20; EO is an ethylene oxide group; PO is a propylene oxide group; x is an integer ranging from 0 to 60; R′ is chosen from optionally substituted divalent hydrocarbons, such as alkylene groups and alkenylene groups comprising from 2 to 22 carbon atoms, and optionally substituted divalent aromatic groups, such as groups of the following formula:

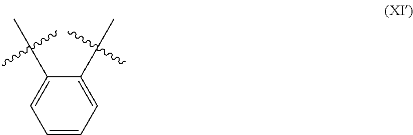 [SMILES: *c1ccccc1*]

and groups of formula

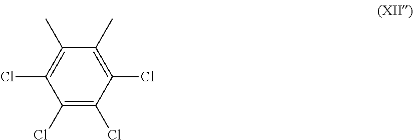

Non-limiting examples of silicone carboxylates include those described in U.S. Pat. Nos. 5,248,783 and 5,739,371, the disclosures of which are incorporated herein by reference, and which are silicone compounds of formula (XII″).

Cationic Polymers Containing at Least One Carboxyl Group.

The cationic polymer can have a negative charge but remains cationic overall, can be an amphoteric polymer that can carry a cationic charge based on pH, or can be a betaine polymer that remains amphoteric at any pH.

The cationic polymers are polymers that result from the homopolymerization or copolymerization of ethylenically unsaturated monomers chosen from: (i) at least one nonionic monomer such as (Alkyl)(Meth)Acrylamide, (Alkyl)(Meth)Acrylate Ester, Vinyl Pyrrolidone, Vinyl Imidazole; (ii) at least one cationic monomer such as Ethyltrimonium (Alkyl)(Meth)Acrylamide, Ethyltrimonium (Alkyl)(Meth)Acrylate Ester, Vinylimidazoline, Dimethylaminopropyl (Alkyl)(Meth)Acrylamide, Methacrylamidopropyl Triethyl Ammonium Chloride (MAPTAC), Diallyl Dimethyl Ammonium Chloride (DADMAC); (iii) at least one (Alkyl)Acrylic acid; (iv) at least one amphoteric monomer such as a carboxybetaine zwitterionic monomer.

Suitable examples of such cationic polymers are: the diallyidimethylammonium chloride/acrylic acid copolymers sold under the names MERQUAT 280 POLYMER or MERQUAT 280NP POLYMER or MERQUAT 281 POLYMER or MERQUAT 295 POLYMER, by the company Nalco (Lubrizol) (INCI name: Polyquaternium-22); the copolymer of methacrylamidopropyltrimonium chloride, of acrylic acid and or methyl acrylate, sold under the name MERQUAT 2001 POLYMER OR MERQUAT 2001N POLYMER by the company Nalco (Lubrizol) (INCI name: Polyquaternium-47); the acrylamide/dimethyldiallylammonium chloride/acrylic acid terpolymer sold under the name MERQUAT 3330DRY POLYMER or MERQUAT 3330PR POLYMER or MERQUAT 3331PR POLYMER or MERQUAT 3940 POLYMER or MERQUAT PLUS 3330 POLYMER OR MERQUAT PLUS 3331 POLYMER by the company Nalco (Lubrizol) (INCI name: Polyquaternium-39); an ampholytic terpolymer consisting of methacrylamidopropyl trimethyl ammonium chloride (MAPTAC), acrylamide and acrylic acid, sold under the name MERQUAT 2003PR POLYMER by the company Nalco (Lubrizol) (INCI name: Polyquaternium-53); Polyquaternium-30, Polyquaternium-35, Polyquaternium-45, Polyquaternium-50, Polyquaternium-54; Polyquaternium-57; Polyquaternium-63; Polyquaternium-74; Polyquaternium-76; Polyquaternium-86; Polyquaternium-89; Polyquaternium-95; Polyquaternium-98, Polyquaternium-104; Polyquaternium-111; Polyquaternium-112, and mixtures thereof.

Latex Polymers

According to various exemplary embodiments, the compositions of the present invention can further comprise one or more latex polymers (also referred to as “latex polymers” in this application) can be chosen from carboxyl functional acrylate latex polymers, carboxyl functional polyurethane latex polymers, carboxyl functional silicone latex polymers, carboxyl functional non-acrylate latex polymers and mixtures thereof.

In various embodiments, the latex polymers of the present invention can be film-forming latex polymers or non film-forming latex polymers.

In at least certain embodiments of the disclosure, the latex polymers are provided in the form of aqueous dispersions prior to formulating the compositions of the disclosure. In various embodiments, the aqueous dispersions may be obtained through an emulsion polymerization of monomers wherein the resulting latex polymers have a particle size lower than about 1 micron. In at least one exemplary embodiment, a dispersion prepared by the polymerization in water of one or more monomers having a polymerizable double bond may be chosen. In another exemplary embodiment, the aqueous dispersions obtained through an emulsion polymerization may be spray-dried.

In other embodiments, the latex polymers are produced from condensation reactions between monomers and subsequently dispersed in an aqueous medium.

Thus, the latex polymers may, in various exemplary embodiments, exist as dispersed polymer particles in a dispersion medium, such as an aqueous dispersion medium. The latex polymers may, in various embodiments, each be dispersed in independent dispersion media or dispersed together in the same dispersion medium.

The dispersion medium comprises at least one solvent chosen from water. The dispersion medium may further comprise at least one solvent chosen from cosmetically acceptable organic solvents such as those described above.

In embodiments according to the disclosure, the latex polymer particles are not soluble in the solvent of the dispersion medium, i.e. are not water soluble and/or are not soluble in the at least one cosmetically acceptable organic solvent. Accordingly, the latex polymers retain their particulate form in the solvent or solvents chosen.

In at least certain exemplary embodiments, latex polymer particles according to the disclosure may have an average diameter ranging up to about 1000 nm, such as from about 50 nm to about 800 nm, or from about 100 nm to about 500 nm. Such particle sizes may be measured with a laser granulometer (e.g. Brookhaven BI90).

In various embodiments, the latex polymers may, independently, be neutralized, partially neutralized, or unneutralized. In exemplary embodiments where the latex polymers are neutralized or partially neutralized, the particle size may be, for example, greater than about 800 nm. In at least certain embodiments, the particulate form of the latex polymers is retained in the dispersion medium.

In further embodiments, the latex polymers may be chosen from uncharged and charged latex polymers. Thus, the latex polymers may, according to various exemplary embodiments, be chosen from nonionic latex polymers, cationic latex polymers, anionic latex polymers and amphoteric latex polymers.

By way of non-limiting example only, the latex polymers may be chosen from carboxyl functional acrylate latex polymers, such as those resulting from the homopolymerization or copolymerization of ethylenically unsaturated monomers chosen from vinyl monomers, (meth)acrylic monomers, (meth)acrylamide monomers, mono- and dicarboxylic unsaturated acids, esters of (meth)acrylic monomers, and amides of (meth)acrylic monomers. The term “(meth)acryl” and variations thereof, as used herein, means acryl or methacryl.

The (meth)acrylic monomers may be chosen from, for example, acrylic acid, methacrylic acid, citraconic acid, itaconic acid, maleic acid, fumaric acid, crotonic acid, and maleic anhydride. The esters of (meth)acrylic monomers may be, by way of non-limiting example, C1-C8 alkyl (meth)acrylates such as methyl (meth)acrylate, ethyl (meth)acrylate, propyl (meth)acrylate, isopropyl (meth)acrylate, butyl (meth)acrylate, tert-butyl (meth)acrylate, pentyl(meth) acrylate, isopentyl (meth)acrylate, neopentyl (meth)acrylate, hexyl (meth)acrylate, isohexyl (meth)acrylate, 2-ethylhexyl (meth)acrylate, cyclohexyl (meth)acrylate, isohexyl (meth)acrylate, heptyl (meth)acrylate, isoheptyl (meth)acrylate, octyl (meth)acrylate, isooctyl (meth)acrylate, allyl (meth)acrylate, and combinations thereof. The amides of (meth)acrylic monomers can, for example, be made of (meth)acrylamides, and especially N-alkyl (meth)acrylamides, in particular N—(C1-C12) alkyl (meth)acrylates such as N-ethyl (meth)acrylamide, N-t-butyl (meth)acrylamide, N-t-octyl (meth)acrylamide, N-methylol (meth)acrylamide and N-diacetone (meth)acrylamide, and any combination thereof.

The vinyl monomers can include, but are not limited to, vinyl cyanide compounds such as acrylonitrile and methacrylonitrile; vinyl esters such as vinyl formate, vinyl acetate, vinyl propionate, vinyl neodecanoate, vinyl pivalate, vinyl benzoate and vinyl t-butyl benzoate, triallyl cyanurate; vinyl halides such as vinyl chloride and vinylidene chloride; aromatic mono- or divinyl compounds such as styrene, a-methylstyrene, chlorostyrene, alkylstyrene, divinylbenzene and diallyl phthalate, as well as para-styrensulfonic, vinylsulfonic, 2-(meth)acryloyloxyethylsulfonic, 2-(meth)acrylamido-2-methylpropylsulfonic acids, and mixtures thereof.

The list of monomers given is not limiting, and it should be understood that it is possible to use any monomer known to those skilled in the art which includes acrylic and/or vinyl monomers (including monomers modified with a silicone chain).

In at least certain, non-limiting exemplary embodiments carboxyl functional acrylate latex polymers may be chosen from aqueous dispersions of Methacrylic Acid/Ethyl Acrylate copolymer (INCI: Acrylates Copolymer, such as LUVIFLEX® SOFT by BASF), PEG/PPG-23/6 Dimethicone Citraconate/C10-30 Alkyl PEG-25 Methacrylate/Acrylic Acid/Methacrylic Acid/Ethyl Acrylate/Trimethylolpropane PEG-15 Triacrylate copolymer (INCI: Polyacrylate-2 Crosspolymer, such as FIXATE SUPERHOLD™ by Lubrizol), Styrene/Acrylic copolymer (such as Acudyne Shine by Dow Chemical), Ethylhexyl Acrylate/Methyl Methacrylate/Butyl Acrylate/Acrylic Acid/Methacrylic Acid copolymer (INCI: Acrylates/Ethylhexyl Acrylate Copolymer, such as Daitosol 5000SJ, Daito Kasei Kogyo), Acrylic/Acrylates Copolymer (INCI name: Acrylates Copolymer, such as DAITOSOL 5000AD, Daito Kasei Kogyo), Acrylates Copolymers, such as those known under the tradenameDermacryl AQF (Akzo Nobel), under the tradename LUVIMER® MAE (BASF), or under the tradename BALANCE CR (AKZO NOBEL), Acrylates/Hydroxyesters Acrylates Copolymer, known under the tradename ACUDYNE 180 POLYMER (Dow Chemical), Styrene/Acrylates Copolymer, known under the tradename Acudyne Bold from Dow Chemical, Styrene/Acrylates/Ammonium Methacrylate Copolymer, known under the tradename SYNTRAN PC5620 CG from Interpolymer, and mixtures thereof.

In yet further exemplary and non-limiting embodiments, the latex polymers may be chosen from carboxyl functional polyurethane latex polymers, such as aqueous polyurethane dispersions. These polyurethanes are conventionally formed by the reaction of prepolymer (i) with a coreactant (ii) to produce a carboxyl terminated or pendant polyurethane polymer. The prepolymer (i) may have the structure according to the formula (I″):

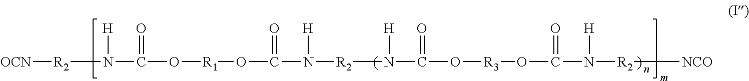

wherein R1 is chosen from bivalent radicals of a dihydroxyl functional compound, R2 is chosen from hydrocarbon radicals of an aliphatic or cycloaliphatic polyisocyanate, and R3 is chosen from radicals of a low molecular weight diol, optionally substituted with ionic groups or potential ionic groups, n ranges from about 0 to about 5, and m is greater than about 1.

Suitable dihydroxyl compounds for providing the bivalent radical R1 include those having at least two hydroxy groups, and having number average molecular weights ranging from about 700 to about 16,000, such as, for example, from about 750 to about 5000. Non-limiting examples of the high molecular weight compounds include polyester polyols, polyether polyols, polyhydroxy polycarbonates, polyhydroxy polyacetals, polyhydroxy polyacrylates, polyhydroxy polyester amides, polyhydroxy polyalkadienes and polyhydroxy polythioethers. In various embodiments, polyester polyols, polyether polyols, and polyhydroxy polycarbonates may be chosen. Mixtures of such compounds are also within the scope of the disclosure.

Optional polyisocyanates for providing the hydrocarbon-based radical R₂ include, for example, organic diisocyanates having a molecular weight ranging from about 100 to about 1500, such as about 112 to about 1000, or about 140 to about 400.

Optional diisocyanates are those chosen from the general formula R₂(NCO)₂, in which R₂ represents a divalent aliphatic hydrocarbon group comprising from about 4 to 18 carbon atoms, a divalent cycloaliphatic hydrocarbon group comprising from about 5 to 15 carbon atoms, a divalent aromatic hydrocarbon group comprising from about 7 to 15 carbon atoms, or a divalent aromatic hydrocarbon group comprising from about 6 to 15 carbon atoms.

The use of diols, for example low molecular weight diols, R3, may in at least certain embodiments allow a stiffening of the polymer chain. The expression “low molecular weight diols” means diols having a molecular weight ranging from about 50 to about 800, such as about 60 to 700, or about 62 to 200. They may, in various embodiments, contain aliphatic, alicyclic, or aromatic groups. In certain exemplary embodiments, the compounds contain only aliphatic groups. The diols that may be chosen may optionally have up to about 20 carbon atoms, and may be chosen, for example, from ethylene glycol, diethylene glycol, propane-1,2-diol, propane-1,3-diol, butane-1,4-diol, 1,3-butylene glycol, neopentyl glycol, butylethylpropanediol, cyclohexanediol, 1,4-cyclohexanedimethanol, hexane-1,6-diol, bisphenol A (2,2-bis(4-hydroxyphenyl)propane), hydrogenated bisphenol A (2,2-bis(4-hydroxycyclohexyl)propane), and mixtures thereof. For example, R3 may be derived from neopentyl glycol.

Optionally, the low molecular weight diols may contain ionic or potentially ionic groups. Suitable low molecular weight diols containing ionic or potentially ionic groups may be chosen from those disclosed in U.S. Pat. No. 3,412,054. In various embodiments, compounds may be chosen from dimethylol-butanoic acid (DMBA), dimethylolpropionic acid (DMPA), and carboxyl-containing caprolactone polyester diol. If low molecular weight diols containing ionic or potentially ionic groups are chosen, they may, for example, be used in an amount such that less than about 0.30 meq of —COOH is present per gram of polyurethane in the polyurethane dispersion. In at least certain exemplary and non-limiting embodiments, the low molecular weight diols containing ionic or potentially ionic groups are not used.

Coreactants (ii) are compounds containing functional groups such as hydroxy or amine groups, in some embodiments primary amine, adapted to react with isocyanate groups in preference to the carboxyl group according to the formula (II″):

X—R4-XX═OH,NH2, (II″)

wherein R₄ represents a divalent aliphatic or cycloaliphatic or aromatic hydrocarbon group, optionally substituted with ionic groups or potentially ionic groups. In various embodiments, compounds may optionally be chosen from alkylene diamines, such as hydrazine, ethylenediamine, propylenediamine, 1,4-butylenediamine and piperazine; In various embodiments, compounds may optionally be chosen from alkylene diols, such as ethylene glycol, 1,4-butanediol (1,4-BDO or BDO), 1,6-hexanediol.

As used herein, ionic or potentially ionic groups may include groups comprising ternary or quaternary ammonium groups, groups convertible into such groups, carboxyl groups, carboxylate groups, sulphonic acid groups, and sulphonate groups. At least partial conversion of the groups convertible into salt groups of the type mentioned may take place before or during the mixing with water. Special compounds may be chosen from dimethylolbutanoic acid (DMBA), dimethylolpropionic acid (DMPA), or carboxyl functional polyester comprising excess equivalents of dicarboxylic acid reacted with lesser equivalents of glycol or carboxyl-containing caprolactone polyester diol.

R1, R2, R3, R4 can have at least one carboxyl group independently.

By way of non-limiting example, such latexes include, but are not limited to, aqueous polyurethane dispersion of Isophthalic Acid/Adipic Acid/Hexylene Glycol/Neopentyl glycol/Dimethylolpropanoic Acid/Isophorone Diisocyanate copolymer (INCI name: Polyurethane-1, such as LUVISET® P.U.R, BASF), a copolymer of hexylene glycol, neopentyl glycol, adipic acid, saturated methylene diphenyldiisocyanate and dimethylolpropanoic acid monomers (INCI name: polyurethane 2), a copolymer of PPG-17, PPG-34, isophorone diisocyanate and dimethylolpropanoic acid monomers (INCI name: polyurethane 4), a copolymer of isophthalic acid, adipic acid, hexylene glycol, neopentyl glycol, dimethylolpropanoic acid, isophorone diisocyanate and bis-ethylaminoisobutyl-dimethicone monomers (INCI name: polyurethane 6), Isophorone diisocyanate, cyclohexanedimethanol, dimethylol butanoic acid, polyalkylene glycol and N-methyl diethanolamine copolymer (INCI name: polyurethane 10), Trimethylolpropane, neopentyl glycol, dimethylol propionic acid, polytetramethylene ether glycol and isocyanato methylethylbenzene copolymer (INCI name: polyurethane 12), Isophorone diisocyanate, dimethylol propionic acid, and 4,4′-isopropylidenediphenol reacted with propylene oxide, ethylene oxide and PEG/PPG-17/3 copolymer (INCI name: polyurethane 14), Isophorone diisocyanate, adipic acid, triethylene glycol and dimethylolpropionic acid copolymer (INCI name: polyurethane 15), 2-Methyl-2,4-pentanediol, polymer with 2,2-dimethyl-1,3-propanediol, hexanedioic acid, methylenedicyclohexanediisocyanate and 2,2-di(hydroxymethyl)propanoic acid, hydrolysed, tris(2-hydroxyethyl)amine salts, reaction products with 1,2-ethanediamine (INCI name: polyurethane 17), Polyurethane-27 is a complex polymer that is formed by the reaction of Polyperfluoroethoxymethoxy Difluorohydroxyethyl Ether and isophorone diisocyanate (IPDI) to form a prepolymer. The prepolymer is further reacted with the triethylamine salt of 3-hydroxy-2-(hydroxymethyl)-2-methyl-1-propionic acid (INCI name: polyurethane 27), a complex polymer formed by reacting dimethylolpropionic acid and a polyester composed of Adipic Acid, Hexylene Glycol, Neopentyl Glycol with methylene dicyclohexyldiisocyanate (SMDI) to form a prepolymer. The prepolymer is neutralized with triethylamine and then chain-extended with hydrazine (INCI name: polyurethane 33).

Carboxylic acid compound chosen from fatty acids, their salts, and mixtures thereof.

The carboxylic acid compound may generally be chosen from saturated or unsaturated carboxylic acids having carbon chains containing from 6 to 30 carbon atoms, in some embodiments from 9 to 30 carbon atoms, and in some embodiments from 9 to 22 carbon atoms and wherein the carbon chain is optionally substituted, for example with one or more (in particular 1 to 4) hydroxyl groups. If the fatty acids of the present disclosure are unsaturated, these compounds may comprise one to three conjugated or unconjugated carbon-carbon double bonds.

Suitable examples of the carboxylic acid compound of the present disclosure are oleic acid, linoleic acid, linolenic acid, isostearic acid, caproic acid, capric acid, caprylic acid, oleic acid, linoleic acid, linolenic acid, behenic acid, lauric acid, myristic acid, stearic acid, palmitic acid and mixtures thereof.

The carboxylic acid compound of the present disclosure may also be chosen from salts of fatty acids, in particular, alkali metal salts of fatty acids (metal soaps) and organic base salts of fatty acids.

The metal of the alkali metal salts of fatty acids includes sodium, potassium, lithium and their mixtures. The organic base salts of fatty acids may be obtained from the neutralization of fatty acids with organic bases such as ammonia, monoethanolamine or triethanolamine. Suitable examples include sodium stearate, zinc laurate, magnesium stearate, magnesium myristate, zinc stearate, potassium cocoate ammonium stearate, ammonium oleate, ammonium nonanoate, and their mixtures.

If present in the composition, the above-described additives are generally present in an amount ranging up to about 95% by weight including all ranges and subranges therebetween, based on the total weight of the composition, such as up to about 50%, up to about 40%, up to about 30%, up to about 20%, up to about 15%, up to about 10%, up to about 5%, such as from about 0.001% to about 50%, or from about 0.001% to about 40%, or from about 0.001% to about 30%, or from about 0.001% to about 20%, or from about 0.001% to about 10%, by weight, based on the total weight of the composition.

Needless to say, a person skilled in the art will take care to select this or these optional additional compound(s), and/or the amount thereof, such that the advantageous properties of the composition, according to the invention, are not, or are not substantially, adversely affected by the envisaged addition.

Methods: Preparation and Use

In some embodiments, the compositions of the present disclosure are prepared by combining polycarbodiimide, optional colorant or dye, and amino compound chosen from polyamines and silicone amines, and a solvent.

In other embodiments, the compositions of the present disclosure are prepared by combining the polycarbodiimide, optional colorant or dye, amino compound chosen from polyamines and silicone amines, and a solvent chosen from water, organic solvents, and mixtures thereof.

In yet other embodiments, the compositions of the present disclosure are prepared by combining the polycarbodiimide, optional colorant or dye, amino compound chosen from polyamines and silicone amines, and a solvent comprising water and organic solvents.

Process of Altering the Color of Hair

In some embodiments, the present invention involves altering the color of hair which may be achieved when the color of hair is lifted or lightened and/or when artificial color is deposited onto hair.

Artificial color may be derived from oxidative coloration using oxidative dye precursors, or from direct coloration using direct dyes, or from temporary coloration using temporary colorants such as pigments and natural dyes.

When lifting of the color of hair is desired, the hair treatment compositions of the present invention are capable of being mixed with an oxidizing composition containing at least oxidizing agent.

When oxidative coloration on hair is to be performed, the hair treatment compositions of the present invention additionally contain a colorant chosen from oxidative dye precursors and such compositions are capable of being mixed with an oxidizing composition containing at least oxidizing agent.

The term “mixed” and all variations of this term as used herein refers to contacting or combining or reconstituting or dissolving or dispersing or blending or shaking the hair treatment composition with the oxidizing composition. It can also mean introducing the hair treatment composition to the oxidizing composition. It may also mean placing the hair treatment composition in the same vessel or container as the oxidizing composition.

Thus, the process of altering the color of hair with an oxidizing composition in accordance with the invention comprises applying a composition for altering the color of hair comprising the hair treatment composition and the oxidizing composition of the present invention onto hair. Said composition that is applied onto hair is formed by mixing the hair treatment composition with the oxidizing composition.

The hair treatment composition can be mixed or combined with the oxidizing composition in a ratio by weight of from about 1:0.01 to about 1:10, such as from about 1:0.01 to about 1:0.05, from about 1:0.05 to about 1:0.1, from about 1:0.1 to about 1:0.5, from about 1:0.5 to about 1:1, from about 1:1 to about 1:2, from about 1:2 to about 1:3, from about 1:3 to about 1:4, from about 1:4 to about 1:5, from about 1:5 to about 1:10. Thus, in accordance with the various embodiments, an inventive composition according to the disclosure can be mixed or combined with an oxidizing composition in a ratio, by weight, from 1 to one of about 0.01, 0.02, 0.03, 0.04, 0.05, 0.06, 0.07, 0.08, 0.09, 0.1, 0.2, 03, 0.4, 0.5, 0.6, 0.7, 0.8, 0.9, 1, 2, 3, 4, 5, 6, 7, 8, 9, and 10, including increments and ranges therein and there between from about 1:0.01 to about 1:10.

Upon application of the composition for altering the color of hair comprising the hair treatment composition and the oxidizing composition and after a resting time (leave-on time) on the hair, for example, ranging from about 1 to about 60 minutes, such as from about 5 to about 45 minutes, or such as from about 5 to about 20 minutes, or such as from about 10 to about 20 minutes, or such as of about 20 minutes, the hair is rinsed, optionally washed with shampoo, rinsed again, optionally washed with a hair conditioning composition, and rinsed again, then dried. The shampoo and hair conditioning composition can be any conventional hair shampoo and conditioner products.

In other embodiments, the composition does not comprise a color altering composition, and is applied to the keratinous substrate either before, at the same time as, or after application of a color altering composition. In some embodiments, application before or after is immediately before or after, and in other embodiments the timeframe between application of the inventive formulations and a color altering composition may be minutes, hours or days.

In addition, independently of whether the inventive composition is applied with a color altering composition, the mixture or composition present on the fibers or hair (resulting from the extemporaneous mixing of the hair treatment and oxidizing compositions, or from the successive application of the hair treatment and oxidizing compositions, or from the application of the composition without any colorant or dye) is left in place for a time, generally, from about 1 to about 60 minutes, such as from about 5 to about 45 minutes, or such as from about 5 to about 30 minutes, or such as from about 5 to about 20 minutes, or such as from about 10 to about 20 minutes, or such as of about 20 minutes, or such as at about 10 minutes.

The temperature during the process of altering the color of hair is between room temperature and 80° C. and in some embodiments, between room temperature and 60° C.

An effective amount of the composition is typically from about 0.1 gram to about 50 grams, and in some applications for treatment of hair, in amounts from about 20 to 60 grams, and in yet further embodiments for an abundance of hair in amounts from about 20 to about 80 grams or more. It will thus be appreciated that the amounts applied depend on the amount or volume of keratinous substrate, such as hair, to be treated and may thus fall within lower ranges for small amounts or patches of hair to the higher ranges and beyond for large amounts or patches of hair. Typical applications are to the whole head in the case of treatment of hair. It will be understood that application to the hair typically includes working the composition through the hair.

The hair that has been contacted with the compositions of the present invention may be air-dried and/or further styled or shaped by applying heat on the hair and/or by combing or brushing or running the fingers through the hair. Other shaping tools may be chosen from combs and brushes.

A suitable applicator device is an applicator brush. It will be appreciated that while a brush is an example of a suitable applicator, particularly for hair, other applicators may be used, including but not limited to spray bottles, squeeze bottles, one and two chamber pumps, tubes, combs, and other applicators known in the art.

Instructions for applying a composition of the present invention onto keratin fibers such as hair may appear on the container (such as can, bottle or jar) holding a composition of the present invention or on the box or carton or other packaging comprising the container holding the composition.

The compositions described above are useful for application onto keratinous substrates such as hair on the head of human individuals.

Thus, the compositions of the present invention can be made into various cosmetic products such hair care products, hair styling products and make up products.

The compositions of the present invention can be in the form of an aqueous composition or an emulsion, such as a lotion or cream.

As used herein, the process and composition disclosed herein may be used on the hair that has been or has not been one or more of artificially dyed, pigmented or permed.

As used herein, the process and composition disclosed herein may be used on hair that has been artificially dyed, pigmented or permed, relaxed, straightened or other chemical process.

The compositions according to the disclosure may be prepared according to techniques that are well known to those skilled in the art.

Although the foregoing refers to various exemplary embodiments, it will be understood that the disclosure is not so limited. It will occur to those of ordinary skill in the art that various modifications may be made to the disclosed embodiments and that such modifications are intended to be within the scope of the disclosure. Where an embodiment employing a particular structure and/or configuration is illustrated in the present disclosure, it is understood that the present disclosure may be practiced with any other compatible structures and/or configurations that are functionally equivalent provided that such substitutions are not explicitly forbidden or otherwise known to be impossible to one of ordinary skill in the art.

The following examples are intended to further illustrate the present invention. They are not intended to limit the invention in any way. Unless otherwise indicated, all parts are by weight.

Testing Procedures

Colorimetric Measurements

For measuring the degree of change in the color of hair (e.g. degree of lightening/lifting color or color deposit) after treating the hair, the color of each swatch are measured with a Minolta CM2600d spectrocolorimeter (specular components included, 10 degrees angle, illuminant D65) in the CIEL*a*b* system.

The L*a*b* colorimetric system is a colorimetric system that assigns each color to a position in a spherical color space. In this color space, the brightness is represented by a position in the ordinate (z-axis) direction, the hue is represented by a position in the circumferential direction, and the chroma is represented by a distance from the center axis. The position on the ordinate (z-axis) representing brightness is designated by L*, and the L* value changes from 0 corresponding to black to 100 corresponding to white. The positive direction of the x-axis corresponds to a red direction, the positive direction of the y-axis corresponds to a yellow direction, the negative direction of the x-axis corresponds to a green direction, the negative direction of the y-axis corresponds to a blue direction, and the position on the x-axis is designated by a* of which value changes from −60 to +60 and the position on the y-axis is designated by b* of which value changes from −60 to +60. The hue and chroma are represented by a* value and b* value, respectively.

Two parameters, L and ΔE (delta-E), may be measured. L* represents the intensity of the color, a* indicates the green/red color axis and b* the blue/yellow color axis. The determination of ΔE values is based on L*, a* and b*.

According to this system, the greater the value of L, the lighter or less intense the color. Conversely, the lower the value of L, the darker or more intense the color (this can also indicate greater color deposit when the composition contains colorants).

The ΔL or the difference between the L value for the treated hair versus the L value for the control hair swatch represents a change in the value of L: the more negative the ΔL value is, the darker the color that is deposited on the hair: Δ L=Lt (treated hair)−Lc (control hair)

Delta-E (ΔE) represents color change. If ΔE is less than 1.0 there is hardly any color difference that the human eye can see. If ΔE greater than 1.0, then there is a noticeable color difference.

### Example 1: Hair Color Protection Using Polycarbodiimide and Polyamine

Objective

Use of Polycarbodiimide and Polyamine as an in-color treatment to improve the color deposit and the color retention of colored hair against shampoos.

Materials

Polycarbodiimide (Carbodilite V-10 from Nishinbo)

Vinylamine/Vinylformamide Copolymer (Lupamine 9080 from BASF)

Protocol

Normal bleach hair swatches (about 5 g) were colored with L'Oreal Excellence 6RR color cream (Intense Auburn) for 30 minutes following the following protocols:

| Protocol | Components |
|---|---|
| 1 | Excellence 6RR only |
| 2 | Excellence 6RR with 2% Polycarbodiimide |
| 3 | Excellence 6RR with 4% Polyamine |
| 4 | Excellence 6RR with 2% Polycarbodiimide + 4% Polyamine |

The following ratio was used for the coloring process:

1 (g of hair):2 (g of color cream/dye):3 (g of 20 Vol developer):0.5 (g of water or material tested), where:

| Protocol | Ratios |
|---|---|
| 1 | the ratio is 1:2:3:0.5 (g of water) |
| 2 | the ratio is 1:2:3:0.25 (g of water):0.25 (g of 2% solution of |
| | Polycarbodiimide) |
| 3 | the ratio is 1:2:3:0.25 (g of water):0.25 (g of 4% solution of |
| | Polyamine) |
| 4 | the ratio is 1:2:3:0.25 (g of 2% solution of |
| | Polycarbodiimide):0.25 (g of 4% solution of Polyamine) |

After coloring, the hair was rinsed with water for 30 seconds, blow dried and shampooed 10 times.

The L, a, b values were taken before and after the shampoos. The change in the total color, dE (delta-E), was then calculated.

Results

| Protocol | a Value (after color) | dE (after 10 shampoos) |
|---|---|---|
| 1 | 14.4 | 13.9 |
| 2 | 15.0 | 13.4 |
| 3 | 15.2 | 14.4 |
| 4 | 15.8 | 12.3 |

The results indicate that, compared to the untreated and the other controls, the swatch treated with color cream containing both Polycarbodiimide and Polyamine shows more red (highest a value) and retains the most color after shampoos (lowest dE).

Specifically, when Polycarbodiimide alone is added to the color composition there is no improvement in color protection as demonstrated by similar dE values. The polyamine alone decreases the color protection as demonstrated by a larger dE value compared to Protocol 1. Only the association of the polyamine and polycarbodiimide provide color protection as demonstrated by a lower dE value from Protocol 1.

The color protection provided by the association is visible to the eye as it is truer to the original color in comparison to all control swatches.

### Example 2: Post Color Treatment for Hair Color Protection Using Polycarbodiimide and Silicone Amine

Objective

Use of Polycarbodiimide and Silicone amine as a post-color treatment to help the color retention of colored hair against shampoos.

Materials

Polycarbodiimide (Carbodilite V-02-L2 from Nishinbo)

Amodimethicone/Silicone Amine (Shin Etsu KF 8020)

Protocol

Normal bleach hair swatches (about 1 g each) were colored with L'Oreal Excellence 6RR color cream (Intense Auburn) for 30 minutes, rinsed and air dried. The following ratio was used for the coloring process:

1 (g of hair):1.5 (g of color cream):3 (g of 20 Vol developer)

After coloration, 2 g of the following treatments were applied on the colored hair, allowed to sit on the hair for 15 minutes and then the hair was blow dried:

| Protocol | Treatments |
|---|---|
| 1 | 50 wt % Water + 50 wt % Isododecane |
| 2 | 10 wt % Polycarbodiimide in Water |
| 3 | 10 wt % Silicone Amine in Isododecane |
| 4 | 5 wt % Polycarbodiimide in Water + 5 wt % Silicone Amine |
| | in Isododecane |

After treatment, the hair was shampooed 10 times.

The L, a, b values were taken before and after the shampoos. The change in the total color, dE (delta-E), was then calculated.

### Results

| Protocol | dE (after 10 shampoos) |
|---|---|
| 1 | 7.55 |
| 2 | 8.74 |
| 3 | 4.05 |
| 4 | 3.52 |

The results indicate that, compared to the untreated and the other controls, the swatch treated with both Polycarbodiimide and Silicone amine retains the most color after shampoos (lowest dE). Specifically, when carbodililte alone is used there is a decrease in color protection as demonstrated by a higher dE value. The silicone amine alone does provide some color protection on its own as demonstrated by a lower dE value. However, the association of the silicone amine and polycarbodiimide provide the best color protection as demonstrated by the lowest dE value.

The color protection provided by the association is visible to the eye as it is truer to the original color in comparison to all controls swatches.

It is to be understood that the foregoing describes some embodiments of the disclosure and that modifications may be made therein without departing from the spirit or scope of the disclosure as set forth in the claims.

What is claimed is:

1. A cosmetic composition for application to a keratinous substrate, comprising:
at least one polycarbodiimide compound; and
at least one amino compound comprising selected from the group consisting of polyamines and silicone amines;
wherein the at least one polycarbodiimide compound and the at least one amino compound are present in at a ratio, by weight, in the range from about 10:1 to about 1:10 of polycarbodiimide to amino compound; and
wherein the composition includes amounts of each of the polycarbodiimide compound and the amino compound sufficient to impart one or more of improved color deposit, improved color retention and extended color durability as compared with compositions comprising only one of the polycarbodiimide and amino compound.

2. A composition for application to a keratinous substrate according to claim 1, wherein the composition comprises from about 0.1% to about 40.0%, by active weight, of the composition, of a combined amount of the polycarbodiimide and the amino compounds.

3. A composition for application to a keratinous substrate according to claim 1, wherein the polycarbodiimide compound comprises a carbodiimide polymer or co-polymer has the following formula: 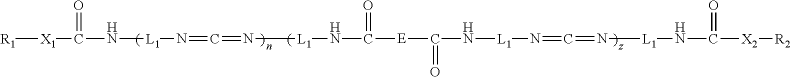
wherein X₁ and X₂, each independently, represents O, S or NH; R₁ and R₂, each independently, a hydrocarbon group containing one or more catenary or non-catenary hetero-atoms and containing linear or branched and cyclic or acyclic groups which are ionic or non-ionic segments or a partially or fully fluorinated hydrocarbon group containing one or more catenary or non-catenary hetero-atoms; n and z are, each independently, an integer of 0 to 20; L₁ represents a C₁ to C₁₈ divalent aliphatic hydrocarbon group, a C₃ to C₁₃ divalent alicyclic hydrocarbon group, a C₆ to C₁₄ divalent aromatic hydrocarbon group, a C₃ to C₁₂ divalent heterocyclic group, or a C₆ to C₁₄ divalent aromatic hydrocarbon group that is not chosen from m-tetramethylxylylene, wherein a plurality of L₁ groups may be identical to or different from one another; E is a radical selected from the group consisting of:
O—R₃—O;S—R₄—S; and
R₅—N—R₄—N—R₅;
wherein R₃ and R₄ are, each independently, hydrocarbon radicals that may contain halogen atoms or one or more catenary or non-catenary hetero atoms, including an aromatic, cycloaliphatic, aryl and linear or branched alkyl radical and R₅ is hydrogen or a hydrocarbon radical, the hydrocarbon radical, when present, includes halogen atoms or one or more catenary or non-catenary hetero atoms.

4. A composition for application to a keratinous substrate according to claim 3, wherein the polycarbodiimide compound is a co-polymer derived from alpha-methylstyryl-isocyanates having the following formula: 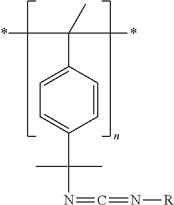
wherein R is an alkyl, cycloalkyl or aryl group having from 1 to 24 carbon atoms.

5. A composition for application to a keratinous substrate according to claim 3, wherein the polycarbodiimide compound is a compound having the following structure: 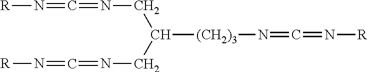
wherein R is an alkyl, cycloalkyl or aryl group.

6. A composition for application to a keratinous substrate according to claim 5, wherein R is selected from the group consisting of an alkyl group having from 1 to 24 carbon atoms, a cycloalkyl group having from 1 to 24 carbon atoms, and an aryl group having from 1 to 24 carbon atoms.

7. A composition for application to a keratinous substrate according to claim 1, wherein the amino compound is selected from polyamines and comprising a colorant, whereby the composition imparts to the keratinous substrate improved color deposit and retention.

8. A composition for application to a keratinous substrate according to claim 1, wherein the composition further comprises a solvent chosen from water, organic solvents, and mixtures thereof.

9. A composition for application to a keratinous substrate according to claim 1, comprising a solvent for the polycarbodiimide selected from distilled or de-ionised water, and a solvent for the amino compound selected from C1-C4 lower alcohols, glycols, polyols, polyol ethers, hydrocarbons, oils, and mixtures thereof.

10. A composition for application to a keratinous substrate according to claim 1, comprising at least one additive chosen from silicone compounds other than the silicone amines of the invention, surfactants (anionic, nonionic, cationic and amphoteric/zwitterionic), and polymers other than the polycarbodiimide of the invention such as anionic polymers, nonionic polymers, amphoteric polymers, polymeric rheology modifiers, thickening and/or viscosity modifying agents, associative or non-associative polymeric thickeners, non-polymeric thickeners, nacreous agents, opacifiers, dyes or pigments, fragrances, mineral, plant or synthetic oils, waxes including ceramides, vitamins, UV-screening agents, free-radical scavengers, antidandruff agents, hair-loss counteractants, hair restorers, preserving agents, pH stabilizers and solvents, and mixtures thereof.

11. A composition for application to a keratinous substrate according to claim 10, wherein the at least one additive comprising one or more rheology modifiers and thickening/viscosity-modifying agents are water-soluble or water-dispersible compounds is selected from acrylic polymers, non-acrylic polymers, starch, saccharide-based polymers (e.g., guar, guar gums), cellulose-based polymers (in particular, hydroxyethylcellulose, cellulose gums, alkyl hydroxyethyl cellulose, carboxylic acid containing celluloses/carbohydrates), non-polymeric and polymeric gelling agents, silica particles, clay, hyaluronic acid, alginic acid, and mixtures thereof.

12. A composition for application to a keratinous substrate according to claim 11, the composition further comprising at least one or more additional compounds selected from polymeric (for example, cellulosic) based thickeners, amphoteric surfactants, nonionic fixing polymers, cationic surfactants, polyols, and propellants.

13. A composition for application to a keratinous substrate according to claim 3, wherein the polycarbodiimide is present in an amount, by weight, based upon the weight of the composition, from about 0.01% to about 20%, and wherein the amino compound selected from a polyamine is present in an amount, by weight, based upon the weight of the composition, from about 0.01% to about 40%, and wherein the amino compound selected from a silicone amine is present in an amount, by weight, based upon the weight of the composition, from about 0.01% to about 40%.

14. A composition for application to a keratinous substrate according to claim 13, wherein the amino compound is a polyamine selected from a vinylamine/vinylformamide copolymer.

15. A composition for application to a keratinous substrate according to claim 13, wherein the amino compound is a silicone amine selected from amodimethicone.

16. A method of treating a keratinous substrate, comprising:
applying to the keratinous substrate a composition of claim 1 in an amount effective to impart one or more of improved color deposit, improved color retention and extended color durability.

17. A method according to claim 16, wherein the application to the keratinous substrate is at least a one step process selected from
a one step process, wherein the composition comprising the polycarbodiimide and the amino compound is provided as a premix, and is prepared by combining the polycarbodiimide and the amino compound and at least a solvent, whereby the mixed composition is applied onto the keratinous substrate;
a one step process, wherein the composition is provided in separate premixes, each premix separately comprising the polycarbodiimide, the amino compound, and the optional colorant or dye, the premixes prepared by separately combining the polycarbodiimide with at least water to form on an aqueous phase, and separately combining the amino compound with at least an organic solvent to form a non-aqueous phase, and separately combining the optional colorant or dye with a suitable solvent, whereby at the time of use, the premixes are combined and agitated to form an emulsion that is applied onto the keratinous substrate;
a two-step process, wherein the composition is provided in separate premixes, the premixes prepared by separately combining the polycarbodiimide with water to form on an aqueous phase, and separately combining the amino compound with an organic solvent to form a non-aqueous phase, and separately combining the optional colorant or dye with a suitable solvent, whereby at the time of use, the premix containing the polycarbodiimide and the premix containing the amino compound are combined to form a mixture, whereby each of the mixture and the premix containing the optional colorant or dye is applied separately to the keratinous substrate in any order; and
a multi-step process, wherein the composition is provided in separate premixes, the premixes prepared by separately combining the polycarbodiimide with water to form on an aqueous phase, and separately combining the amino compound with an organic solvent to form a non-aqueous phase, and separately combining the optional colorant or dye with a suitable solvent, whereby at the time of use, each of the premixes is applied separately to the keratinous substrate in any order.

18. A method of treating a keratinous substrate, comprising:
applying to the keratinous substrate a composition of claim 1 comprising an amino compound selected from polyamines and also comprising a colorant, the combination in an amount effective to impart one or more of improved color deposit, improved color retention and extended color durability.

19. A method of treating a keratinous substrate, comprising:
applying to the keratinous substrate a composition of claim 1 an amino compound selected from silicone amines in an amount effective to impart one or more of improved color deposit, improved color retention and extended color durability.

20. An article of manufacture comprising a kit containing, in separately packaged form, the kit comprising:
at least one of:
a composition according to claim 1 wherein the polycarbodiimide, the optional colorant or dye, and amino compound are combined and the composition further comprises at least a solvent; and
a composition according to claim 1 wherein the polycarbodiimide, the optional colorant or dye, and amino compound are provided in two or more separate packages,
whereby at the time of use, the contents of the packages are one or more of combined and agitated to form an emulsion, and directly applied to the keratinous substrate.
